(19) United States
(12) Patent Application Publication
(10) Pub. No.: US 2013/0267547 A1
(43) Pub. Date: Oct. 10, 2013

(54) PRODRUGS UTILIZING A TRANSPORTER-DIRECTED UPTAKE MECHANISM

(71) Applicant: VIRGINIA COMMONWEALTH UNIVERSITY, Richmond, VA (US)

(72) Inventors: Phillip M. GERK, Mechanicsville, VA (US); Scott W. Walsh, Chesterfield, VA (US); Meng WANG, Richmond, VA (US); Andrew K. LANDSBERG, Eagle-vail, CO (US)

(21) Appl. No.: 13/834,686

(22) Filed: Mar. 15, 2013

Related U.S. Application Data
(63) Continuation-in-part of application No. PCT/US11/55231, filed on Oct. 7, 2011.
(60) Provisional application No. 61/391,177, filed on Oct. 8, 2010.

(51) Int. Cl. A61K 47/48

(52) U.S. Cl. CPC A61K 47/48061
USPC 514/274; 435/375; 544/316; 549/364

(57) ABSTRACT

Prodrugs comprising a lipophilic drug linked to a transport moiety that can be taken up by a fatty acid transporter are provided. The transport moiety comprises a lipid chain connected to a hydrophilic group (e.g. a carboxylic acid, a phosphate, or a sphingosine-like moiety). Due to the presence of the transport moiety, the prodrugs are substrates for endogenous fatty acid transporter systems. The transport moiety thus serves as a carrier or targeting moiety to facilitate uptake of the entire prodrug complex by endogenous fatty acid transporter systems, thereby moving the prodrug into cells and tissues where drug distribution and effects are desired. Hydrolysis of the chemical linkage between the lipid-like moiety and the lipophilic drug releases the drug in an active form within the cells or tissues.

### CROSS-REFERENCE TO RELATED APPLICATIONS

This application is a continuation-in-part (CIP) application of and claims priority to International Application PCT/US2011/55231 filed Oct. 7, 2011, which claims priority to U.S. Provisional Application 61/391,177 filed Oct. 8, 2010, and the complete contents thereof is herein incorporated by reference.

### BACKGROUND OF THE INVENTION

1. Field of the Invention

The invention relates to the delivery of prodrugs to a desired site of action via uptake by endogenous lipid transport mechanisms. The prodrugs comprise a drug (e.g. a large, lipophilic drug) linked, via a hydrolyzable chemical bond, to a transport moiety that causes the prodrug to be taken up by a fatty acid transporter.

2. Background of the Invention

The development of methods for the targeted delivery of drugs to a site of action in an active form is a desideratum in the medical field that warrants the expenditure of hundreds of millions of dollars each year. Technologies have been developed whereby moieties that enhance solubility, permeability and stability are attached to drugs, e.g. via ester linkages. Recently, patents have issued for transporter directed prodrug approaches which utilize, for example, peptide transporters such as hPEPT1/2, monocarboxylate transporters such as MCT1-4, and multivitamin transporters such as SMVT to enhance uptake of drugs from the gastrointestinal tract. See, for example, U.S. Pat. No. 7,671,082 to Moher, and US patent applications 2003/0158089 to Gallop et al. and 2005/0025839 to Polli, the compete contents of each of which are hereby incorporated by reference. In these cases, a moiety that is readily taken up by a transporter protein and translocated across the cell membrane is attached to a drug of interest, and the entire drug-moiety complex is taken up by the transporter and delivered into the cell. However, these transporters have only a limited capacity to accept large, lipophilic compounds, such as certain promising compounds used in the treatment of cancer and HIV.

United States patent application 20090123388 to Ganaphthy et al. (Prodrugs of Short-Chain Fatty Acids and Treatment Methods) is based on the discovery that the ATB₀+ amino acid transport system can be used to transport prodrugs comprising a neutral or cationic amino acid that has been modified to comprise a short-chain fatty acid moiety, such as butyrate or pyruvate, into affected cells where the short-chain fatty acids exert their beneficial effect. These prodrugs are useful for treatment of colon cancer, inflammatory bowel disease, ulcerative colitis, Crohn's disease, lung cancer, cervical cancer, and cancers resulting from metastases from primary colon cancer sites. However, this prodrug system is also not designed to deliver large, lipophilic drugs.

Sohma et al, (J. Med. Chem. 2003, 46, 4124-4135) describe the development of water soluble prodrugs of the HIV-1 protean inhibitor KNI-727 (amprenavir). The prodrugs comprise both amprenavir and a hydrophilic solubilizing moiety linked to drug via a self-cleaveable spacer. However, this study fails to suggest or take into account potentially advantageous transport mechanisms within cells or tissues.

Matsumoto et al., [Bioorganic & Medicinal Chemistry 9 (2001) 417-430] describe prodrug forms of HIV protease inhibitors. However, this research group reached the conclusion that the presence of free carboxylic acids in the prodrug deterred penetration of the prodrug across the cell membrane. This may have been because they failed to recognize the importance of using a suitably ionizable group in the prodrug, or to take into account potentially advantageous transport mechanisms within cells or tissues.

U.S. Pat. No. 5,914,332 to Sham, et al. describes lopinavir derivatives, but does not show or suggest modifications of lopinavir which facilitate uptake into cells or tissues, or which take into account the time of bioavailabiltiy of the drug.

There is an ongoing need to develop additional drug delivery strategies, particularly for large, lipophilic compounds.

### SUMMARY OF THE INVENTION

The invention expands the realm of transporter-directed prodrug approaches to drug delivery, particularly the delivery of large, lipophilic molecules, by utilizing the previously unexploited fatty acid uptake and transporting system. According to the invention, a drug or molecule of interest is converted into a substrate for uptake by a fatty acid transporter by the attachment of a transport moiety, converting the drug to a prodrug that readily binds to and is taken up by the transporter. The transport moiety, which comprises a lipophilic spacer chain and a hydrophilic group, thus acts as a carrier or targeting moiety for uptake of the entire prodrug structure via the fatty acid transport system. Attachment of the drug to the transport moiety is generally via a chemical bond that is susceptible to hydrolysis. Therefore, once inside a cell or tissue of interest, the transport moiety is cleaved (hydrolyzed) from the prodrug structure, releasing the drug in an active form. In some embodiments, the prodrugs are advantageously ionizable at physiological pH, at levels which provide suitable or reasonable prodrug halflives, having components with a pKa at or below 4.5. Because this prodrug system utilizes fatty acid transporters for uptake, the system is particularly useful for the delivery of drugs, especially large, lipophilic drugs, to areas of the body which were previously difficult to target, for example, the fetal placental unit, the central nervous system, gut-associated lymphatic tissue, the brain, and tumors. The invention may be applied to drug molecules which are intrinsically lipophilic, or modified to become more lipophilic.

### BRIEF DESCRIPTION OF THE DRAWINGS

FIG. 1. Schematic representation of uptake of a prodrug of the invention via a fatty acid transport system into target cells or tissues.

FIG. 2A-I. Structures of exemplary HIV Protease Inhibitors (HIVPI's). A, amprenavir; B, darunavir; C, atazanavir; D, indinavir; E, lopinavir; F, nelfinavir; G, ritonavir; H, saquiinavir; I, tipranavir; Arrows show hydroxyl groups susceptible to conjugation to a lipid-like structure.

FIG. 3A-Y. Exemplary prodrugs. A, succinyl-lopinavir; B, diglycolic (diacetic)-lopinovir; C, thiodiglycolic (thiodiacetic)-lopinavir; D, fumaryl-lopinavir; E, muconyl-lopinavir; F, adipoly-lopinavir; G, thiopropionyl-lopinavir; H, 2-ketoglutaryl (α-ketoglutaryl)-lopinavir; I, 3-ketoglutaryl-lopinavir; J, cyclohexanedioyl-lopinavir; K, glycerosuccinyl-lopinavir; L, generic depiction of lopinavir carbamates, where R=various saturated or unsaturated alkyl or aromatic groups; M, citrosuccinyl-lopinavir; N, malosuccinyl-lopinavir; O, diglycolic-ritonavir; P, diglycolic-saquinavir; Q, diglycolic-nelfinavir; R, diglycolic-nelfinavir; S, diglycolic-atazanavir; T, diglycolic-tipranzvir; U; diglycolic-indinavir; V, diglycolic-indinavir; Wa, 17-diglycolic-estradiol; Wb, 3-diglycolic-estradiol; Xa, 17-diglycolic-2-methoxyestradiol; Xb, 3-diglycolic-2-methoxyestradiol; Ya, 4′-succinic-resveratrol; Yb, 3-succinic-resveratrol.

FIG. 4A-C. Exemplary HPLC results for A, 3-ketoglutaryl-lopinavir, succinyl-lopinavir, thoipropionyl-lopiinavir and adipoyl-lopinavir; B, 3-ketoglutaryl-lopinavir and 2-ketoglutaryl-lopinavir; C, muconyl-lopinavir.

FIGS. 5A and B. Plasma stability of A, succinyl-lopinavir; and B, oxydiacetic-lopinavir.

FIGS. 6A and B. Temperature dependence of succinyl-lopinavir vs lopinavir. A, trial 1 (BeWo cells); B, trial 2 (human primary cytotrophoblast cells).

FIG. 7. Comparison of lopinavir (LPV) and succinyl-lopinavir (SLPV) uptake by cytotrophoblast cells.

FIGS. 8A and B. Comparison of lopinavir (LPV) and succinyl-lopinavir (SLPV) uptake by BeWo cells.

FIGS. 9A and B. Diglycolic-lopinavir data. A, medium containing 100 uM DGLPV before (a) and after (b) 30 minutes of incubation with fresh human placental villous tissue; B, LPV detected in fresh human placental villous tissue after 30 minutes of incubation with 100 uM DGLPV.

FIG. 10. Uptake of ³H-lopinavir (LPV), 3H-succinyl-lopinavir (SLPV), and ³H-carnitine-succinyl-lopinavir (CS-LPV; alone or with unlabelled carnitine 1 mM). BeWo cells (passage 38) were incubated with the compounds for 10 minutes at 37° C. or 4° C. in Dulbecco's phosphate buffered saline containing 0.05% bovine serum albumin. After incubation, the cells were washed with the cold buffer (lacking the compounds), and compounds were extracted from the cells into methanol followed by liquid scintillation analysis. Two-way ANOVA with Bonferroni post tests were used to compare groups; comparisons showed that SLPV uptake was significantly greater than LPV uptake, while CS-LPV uptake was significantly less than LPV uptake regardless of the absence or presence of 1 mM unlabelled carnitine. SLPV uptake at 4° C. was significantly less than at 37° C., indicating a temperature-dependent biological transport process rather than diffusion or non-specific binding.

FIGS. 11 a-b show seven exemplary prodrugs and highlighting the transport moiety with a circle. FIG. 11a shows variations in chain length; FIG. 11b shows variations in electronic properties, with changes in pKa values.

FIGS. 12a-b show exemplary synthesis procedures for preparing the prodrugs set forth in FIGS. 11a-b.

FIG. 13 shows an exemplary synthesis procedure for preparing the prodrug 3, etoposide acetonide hemiglutarate. In this embodiment, a hydrophilic drug is made lipophilic by attaching protecting groups to the hydrophilic functional groups, in addition to the transport moiety.

FIGS. 14A-F. are data and graphs relating to investigations on LPV esters. NMR spectroscopy was performed on the compounds including GLPV (FIG. 14A). The NMR spectrum shows that the chemical environment of an isopropyl group of GLPV no longer permits free rotation, as was seen in LPV spectrum. An LC-MS/MS assay was developed and validated to determine concentrations of the novel compounds in biological matrices and fluids, as shown in FIG. 14B. This assay was used to determine the uptake of non-radiolabelled LPV esters (GLPV, SLPV, and DLPV) in BeWo cells (FIGS. 14C and E), their stability in plasma (FIG. 14D), and their hydrolysis in vivo in rats (FIG. 14F). The results show that uptake of GLPV>SLPV>DGLPV. The figures also show that uptake of GLPV, SLPV, and DGLPV are all temperature dependent, consistent with a fatty acid transporter-mediated uptake mechanism. Finally, the results show that DGLPV and GLPV are capable of being hydrolyzed in vivo.

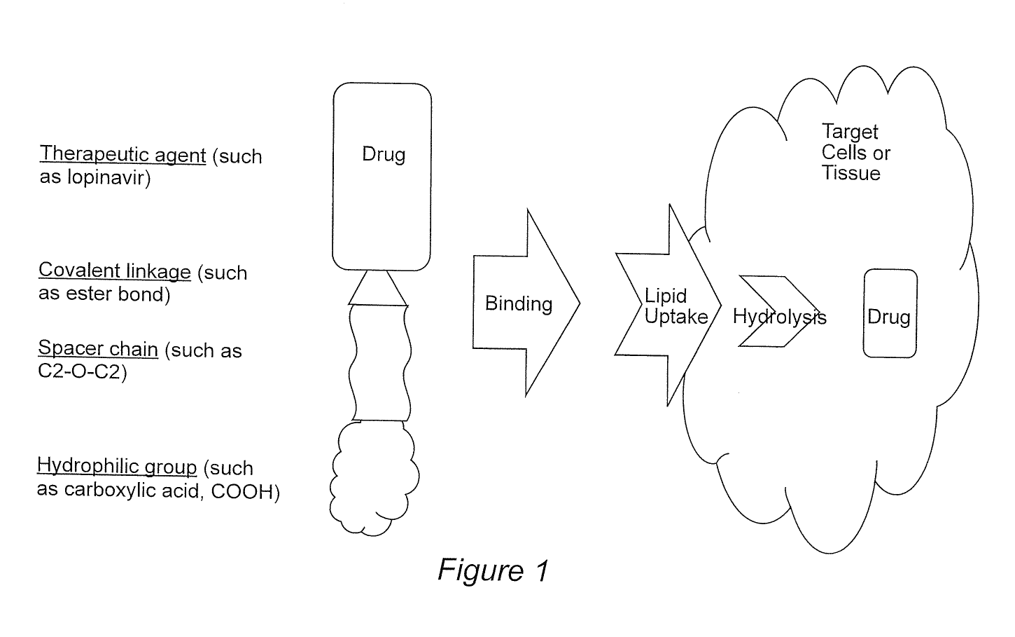

### DETAILED DESCRIPTION

The invention provides prodrugs comprising a drug or molecule of interest which is chemically linked (attached) to a transport moiety which renders the prodrug capable of uptake by endogenous fatty acid transport systems. The transport moiety, in effect, “disguises” the prodrug as a fatty acid transport system substrate, and confers upon the entire prodrug the property of interacting with and being taken up by a fatty acid transport mechanism. The transport moiety thus facilitates recognition and uptake of the prodrug by components (usually one or more proteins) of a fatty acid transport system that is endogenous within living organisms, providing a mechanism of transport across membranes into tissues or cells accessed by the fatty acid transport system. The prodrugs may advantageously permit the administration of lower amounts of a drug, thereby precluding or lessening side effects.

Lipid transport systems accept and transport free fatty acids, (such as long-chain saturated or unsaturated carboxylic acids), phospholipids (such as mono-alkyl phosphoesters), sphingolipids (such as sphingosine), and derivatives of fatty acids (such as numerous arachidonic acid metabolites (prostaglandins, thromboxanes, leukotrienes, etc.)). According to the invention, a relatively lipophilic compound (such as an HIV protease inhibitor, steroid hormone, etc.), which would otherwise not be a substrate for uptake by a fatty acid transport system, is conjugated (through e.g. esterification, amidation, etc.) to a transport moiety as described herein, to form a prodrug compound which resembles a lipid molecule, having a polar head group (such as a free carboxylate) and a non-polar tail (including the drug and a spacer chain of the transport moiety). Such compounds are taken up by fatty acid transport systems and carried into body tissues and cells, and hydrolyzed, e.g. by cellular esterases and/or amidases, thus releasing the active (parent) compound in the target tissue.

By a “fatty acid transport mechanism” or “fatty acid transport system”, we mean the protein facilitated transport (translocation, movement, etc.) of fatty acids across plasma membranes, e.g. across membrane bilayers. Without being bound by theory, data presented herein appear to implicate the FATP4 transport system as the one that by which the prodrugs of the invention are transported into previously unaccessible cells and tissues, particularly those which function as reservoirs of disease causing agent such as viruses.

The prodrug components and various other aspects of the invention are discussed in detail below.

### The Transport Moiety

The prodrugs of the invention comprise a “transport moiety” that, when attached to a substance of interest, converts the substance to a substrate for fatty acid transport systems. As present in the prodrug, the transport moiety generally comprises a “spacer” or “spacer chain” or “spacer element” comprising atoms or groups of atoms that are hydrophobic, and further comprises a hydrophilic group at a terminus of the spacer chain (the end of the spacer that is not linked to the substance of interest). As such, the spacer separates or spaces apart the substance of interest and the hydrophilic group.

Exemplary hydrophobic spacers include but are not limited to: saturated and unsaturated branched or unbranched aliphatic chains which comprise from about 3 to about 20 CH₂ groups (e.g. about 2, 3, 4, 5, 6, 7, 8, 9, 10, 11, 12, 13, 14, 15, 16, 17, 18, 19, or 20 CH₂ methyl groups, usually from about 3 to 18 for example: ethyl, propyl, butyl, pentyl, hexyl, and longer alkyl chains; as well as saturated and unsaturated and branched and unbranched forms of such alkyl chains. Substituted chains are also encompassed, in which one or more CH₂ groups in the chain are substituted by a heterologous atom such as O, N, S, CO, Se, Si, B, etc. In other embodiments, the hydrophobic element is or contains an aromatic moiety, e.g. an aromatic hydrocarbon (i.e. arene, aryl hydrocarbon, etc.), which may be mono- or polycyclic, and may be unsubstituted or substituted with hetero atoms (e.g. O, N, S, CO, etc.), e.g. heterocylics such as furan, pyridine, pyrazine, imidazole, pyrazole, oxazole, thiophene, and their benzannulated analogs (e.g. benzimidazole). In other embodiments, the hydrophobic spacer is or contains a cycloalkyl moiety, which may be substituted or unsubstituted, and which may be mono- or polycyclic, e.g. cyclopropyl, cyclobutyl, cyclopentyl, cyclohexyl, etc. substitutents, and polycycles and heterocycles of these.

The hydrophobic spacer element provides, at one position, a point of attachment for the drug or molecule of interest, usually at one end of the spacer chain. The attachment point is usually a reactive group that is capable of forming a chemical bond (usually a hydrolyzable covalent bond) with a reactive group on the drug or molecule of interest that is to be attached to the transport moiety. Exemplary reactive groups that may form or be part of the attachment point include but are not limited to: hydroxyl, carboxyl, amine, phosphate, carbonate, etc.

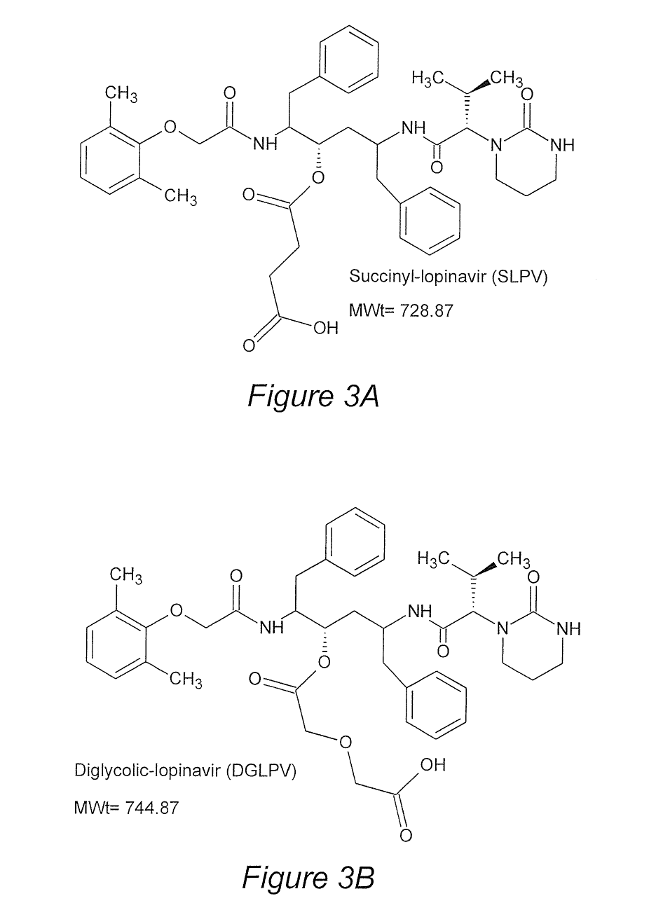

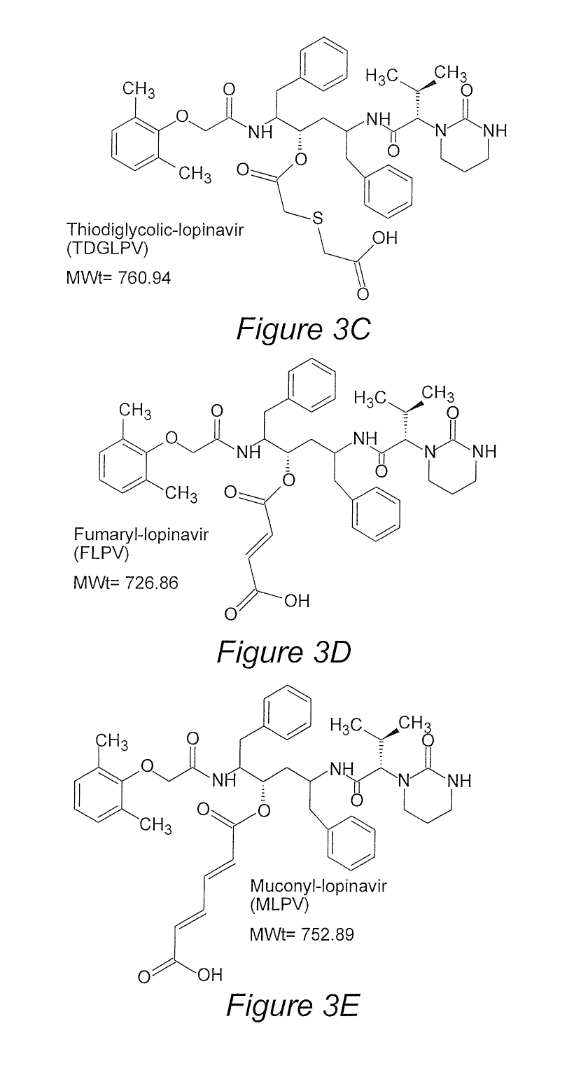

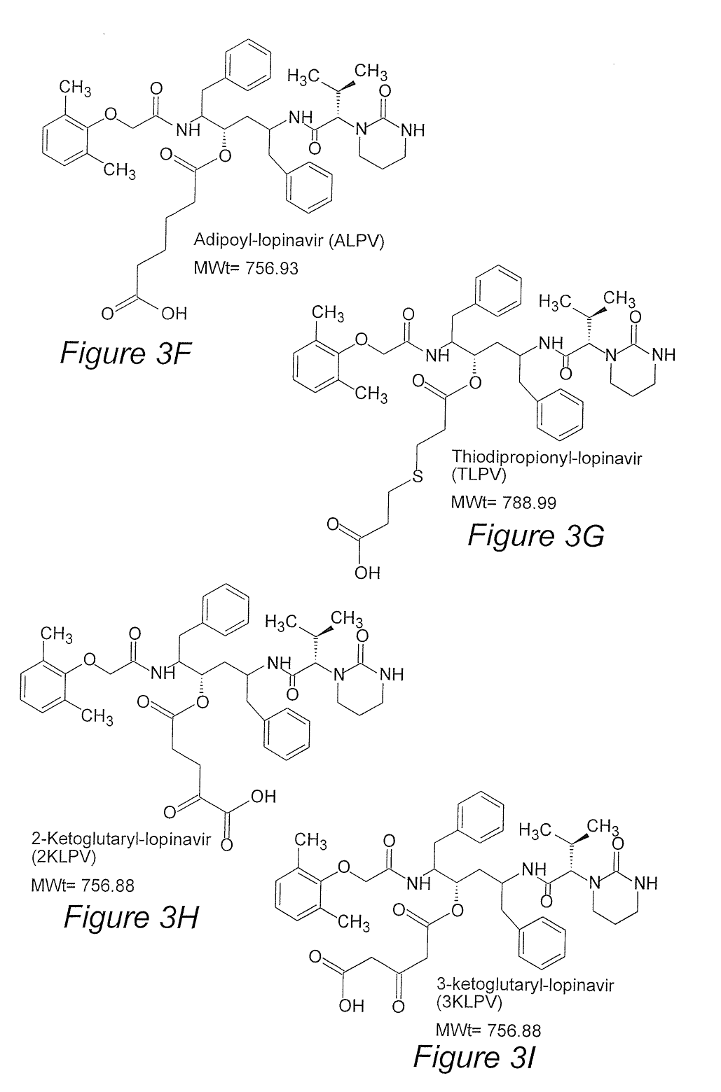

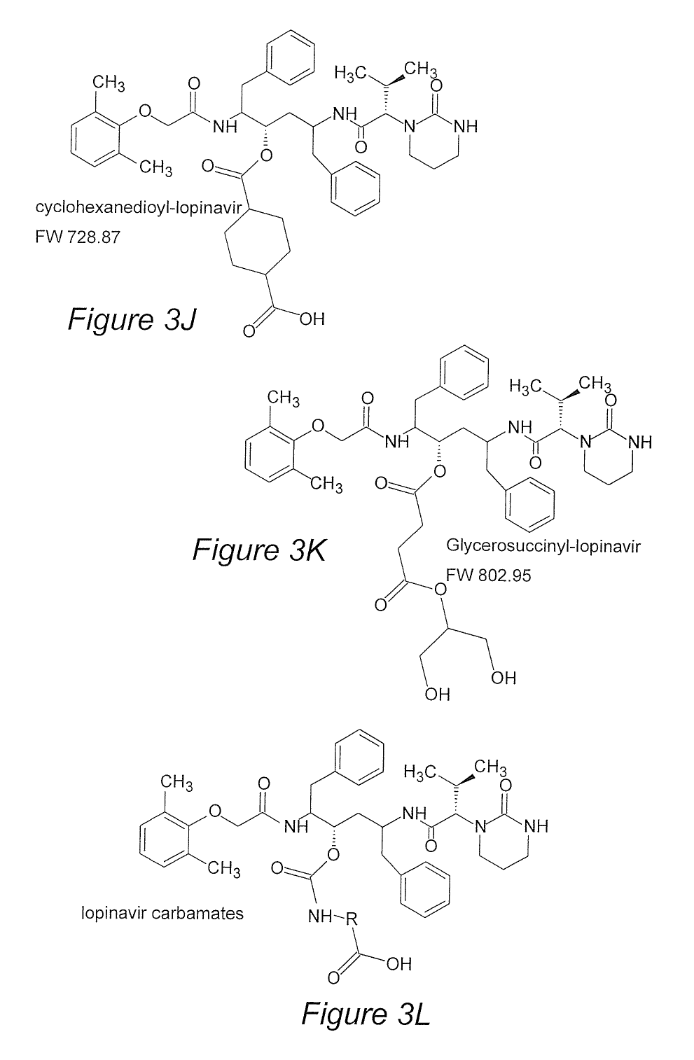

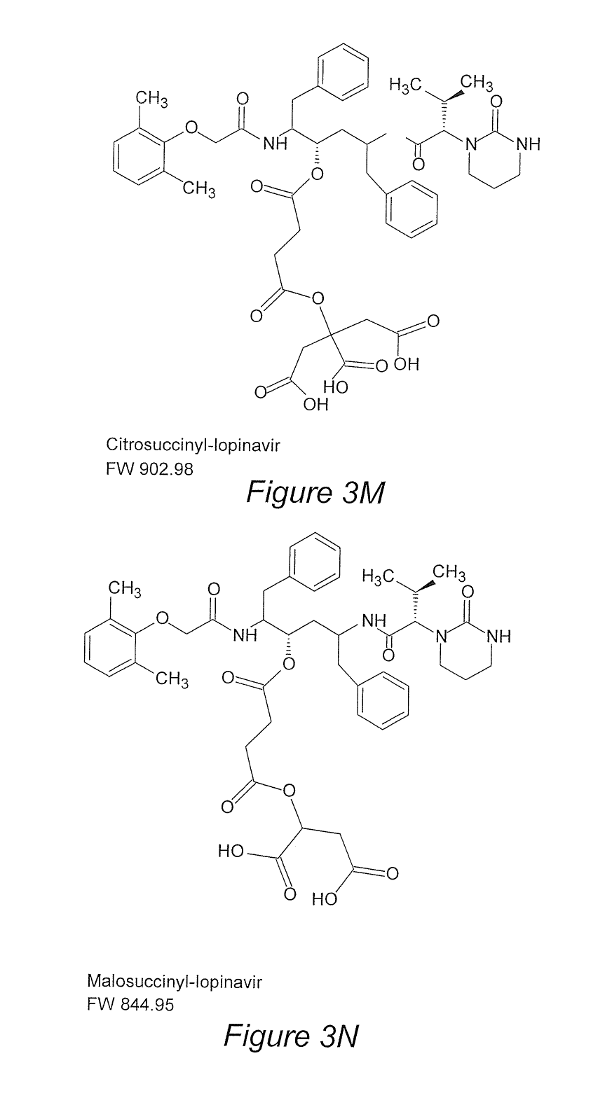

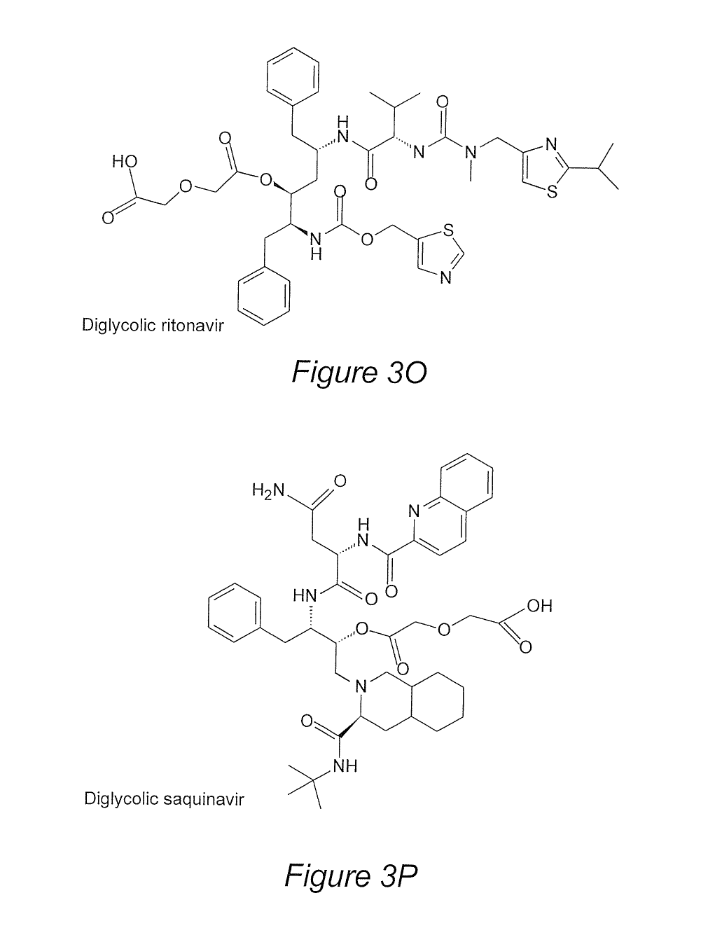

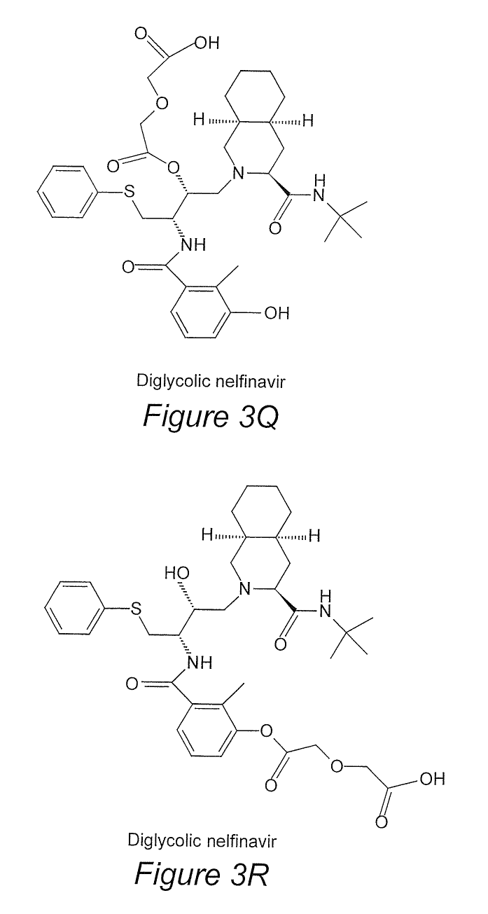

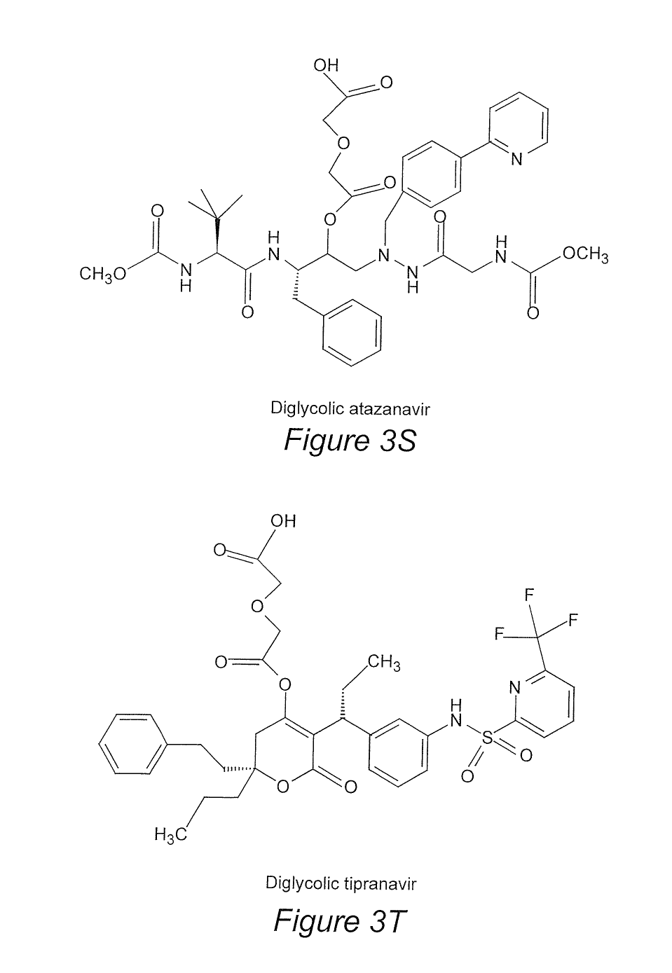

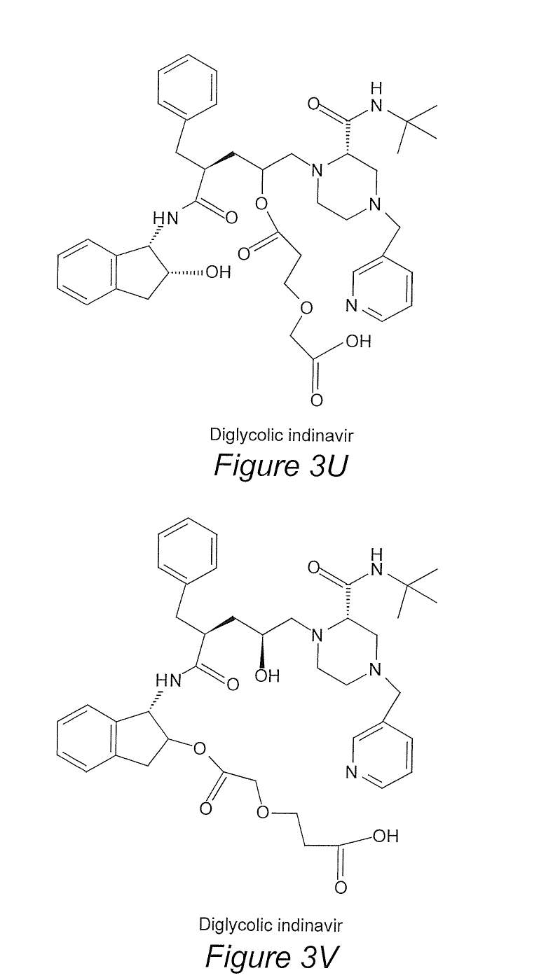

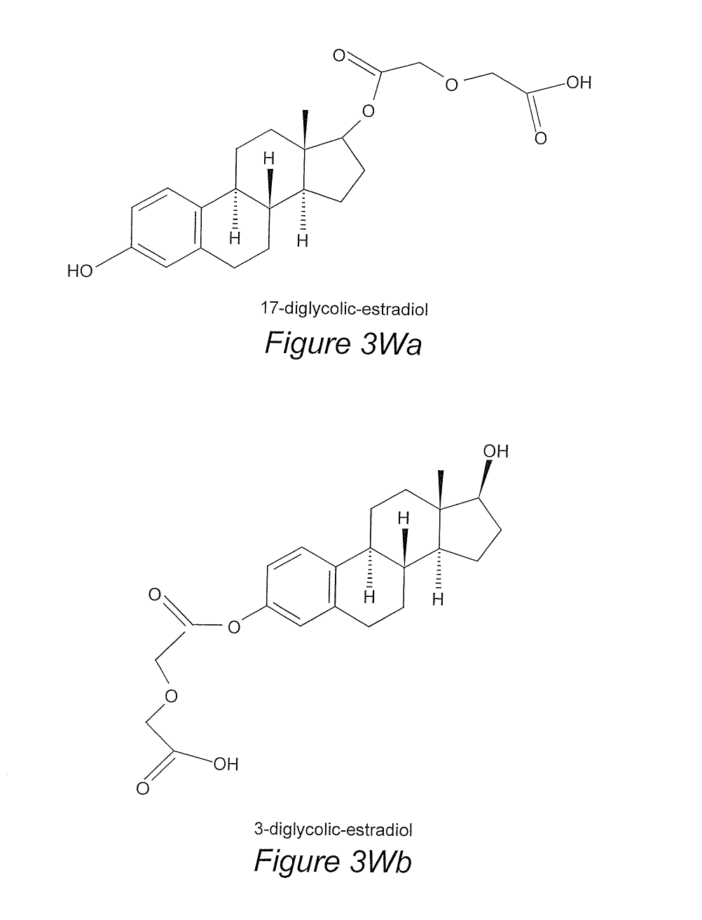

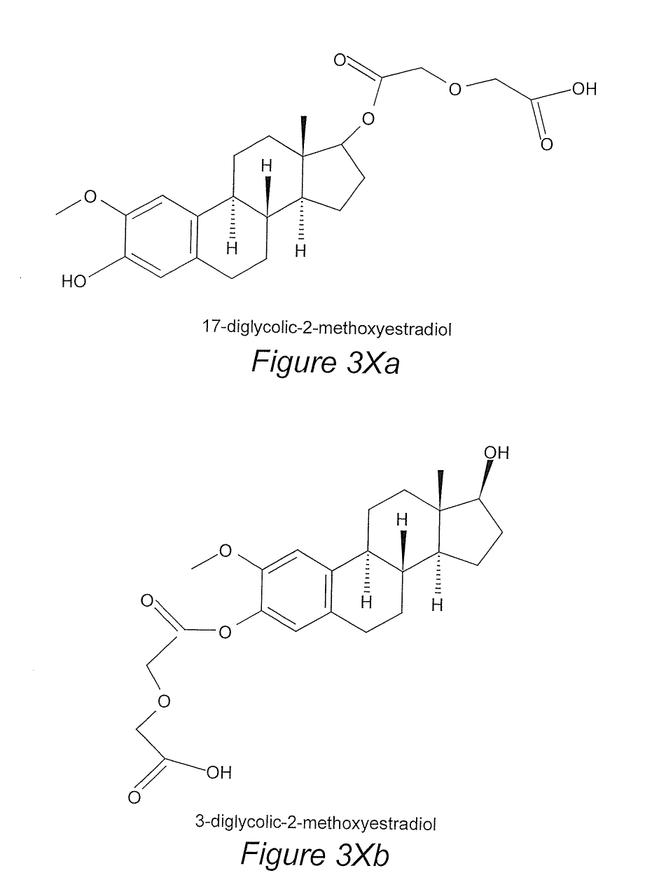

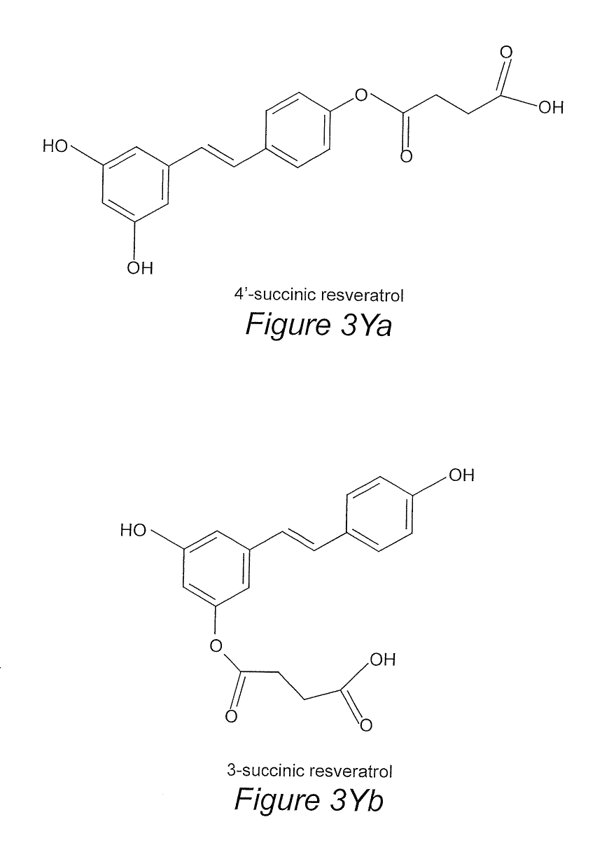

The length of the spacer element and its composition will affect both the stability of the conjugate, the release of the active (parent) compound, and the degree to which the conjugate interacts with lipid transport pathways. A chain length that is too short (e.g. oxalic acid) or too long (e.g. octadecanedioic acid) would be unfavorable. An unsaturated chain may be favorable both for interacting with lipid transporters and for releasing the active compound by hydrolysis. In one embodiment, the length of the spacer is from about 3 to about 18 atoms. The “length” of the spacer is calculated by counting the number of contiguous atoms in the chain (including C atoms and heteroatoms in the chain, e.g. O, S, N, etc.), without including atoms that are bonded directly to the substance of interest (e.g. 0 of an ester linkage is not counted) and without counting the atoms of the hydrophilic group which are adjacent to an ionizable atom (e.g. C of COOH or COO⁻ is not counted). If multiple chains are present in the spacer, the longest chain is used to determine the “length”. If cyclic compounds are present in the spacer, the counting of the atoms proceeds down (through) only the side of the cyclic compound that has the fewest atoms, i.e. along the shortest atomic “path”. For example, succinyl derivatives such as succinyl-lopinavir (FIG. 3A) have a spacer length of 3, diglycolic derivatives (e.g. diglycolic-lopinavir, FIG. 3B) have a spacer length of 4, adipoyl derivatives (adipoyl-lopinavir, FIG. 3F) have a spacer length of 5, glycerolsuccinyl derivatives (e.g. glycerolsuccinyl-lopinavir, FIG. 3K) have a spacer length of 6, cyclohaxanedioyl derivatives (e.g. cyclohexanedioyl-lopinavir, FIG. 3J), have a spacer length of 5, etc.

The hydrophilic group that is attached to the hydrophobic chain may be any hydrophilic group that facilitates uptake of the prodrug by a fatty acid transport system. In some embodiments, the hydrophilic group is, for example, a carboxylate, phosphate, a phosphate, a sphingosine-like moiety, or glycerol, serine, choline, betaine, ethanolamine, taurine, etc. In some embodiments, the hydrophilic group is a carboxylic acid. In some embodiments, the carboxylic acid is a dicarboxylic acid.

The prodrugs of the invention may be tailored with respect to the rates of uptake and hydrolysis of the prodrug by varying the ionization properties of the transport moiety. The hydrophilic group is generally ionizable, at physiological pH (e.g. about 6.5 to about 7.8), and a suitable pKa value of an ionizable atom or group of the hydrophilic groups is generally less than about 4.5 or less, e.g. about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. These values are advisable in order to increase hydrolysis and release of the drug to levels that are physiologically relevant, an aspect of prodrug development that was previously unappreciated.

In some embodiments, the transport moiety is a naturally occurring molecule which inherently contains a hydrophobic element, a hydrophilic group, and a point of attachment for a drug or molecule of interest. In other embodiments, the lipid-like moiety is synthetic or partly synthetic in that it is created by the attachment of one or more chemical groups to each other and/or chemical modification of one or more components, to form the lipid-like moiety. For example, a hydrophilic group of interest may be attached to a hydrophobic element of interest, which is then modified to contain an attachment point; or a naturally occurring molecule which contains a hydrophobic element and a hydrophilic group may be chemically modified to contain a reactive group that serves as a point of attachment for a drug or molecule of interest; or a hydrophobic element that contains a suitable point of attachment may be modified by being joined to a suitable hydrophilic group, etc. Further, in some embodiments, the reactive attachment group and the hydrophilic group may have the same or similar compositions, e.g. both may be carboxylates.

Examples of moieties which contain both a spacer chain and a carboxylic acid hydrophilic group, together with a point of attachment for a substance of interest, and which may be used to modify a lipophilic substance of interest as described herein, include but are not limited to: acids such as oxalic, malonic, succinic, glutaric, adipic, pimelic, suberic, sebacic, fumaric, maleic, aconitic, muconic, dihydromuconic, diglycolic, thiodiglycolic, oxydipropionic, thiodipropionic, 2 ketoglutaric, 3 ketoglutaric, 4-carboxybenzoic, cyclohexanedioic, tetramethylheptanedioic, tetramethylhexanedioic, furandicarboxylic, naphthalic, and thiodiacetic sulfoxide acid. Further examples include carboxylic acids with saturated hydrophobic chains (such as hexadecanedioic acid), unsaturated hydrophobic chains (such as octadecenedioic acid) and polyunsaturated (octadecadienedioic acid), 3,3′-oxydipropionic, 4-carboxybenzoic, tetramethylheptanedioic, cis-aconitic, furandicarboxylic, thiodiacetic acid sulfoxide, dihydromuconic, pimelic, glutaric, suberic, sebacic, dodecanedoic, tetrahydrofuran 2,5-dicarboxylic acid, norcamphoric acid, cyclopentadiene-1,3-dicarboxylic acid, as well as variants of the above having methyl or ethyl branches located between the two carboxylic acid groups, and other dicarboxylic acids of varying chain length and position and degree of unsaturation.

In some embodiments, the hydrophilic group is a lipid mimic such as a sphingosine-1-phosphate derivative, in which the long chain is partly or fully replaced by a link to the therapeutic agent (e.g. the large lipophilic molecules described herein). Also, included would be other variations such as 12-(phosphonooxy)-dodecanoic acid, in which the diacid is linked to the therapeutic agent, e.g. by either a carboxyester or a phosphoester bond.

The prodrugs of the invention may be tailored with respect to the rates of uptake and hydrolysis of the prodrug by varying the nature of the transport moiety. For example, a pKa value of less than about 4.5, and usually lower than about 3 (e.g. about 2.9, 2.8, 2.7, 2.6, 2.5, 2.4 or 2.3 or lower) may be advisable to increase hydrolysis and release of the drug. The rationale is as follows: delivery of the prodrug to the tissue is one issue, but it does little good if the prodrug itself is not active, and if the prodrug moiety fails to release the active compound. For example, the hydrolysis of SLPV has been tested and it has been found that it is completely stable to plasma esterases. Thus, it is unlikely that SLPV itself would be active against HIV, especially based on structural models of HIV protease. Rather, for activity, the drug must be released from the prodrug. However, other dicarboxylates are more susceptible to plasma and tissue esterases, as well as to chemical hydrolysis, for example, dicarboxylates with pKa values lower than succinic acid, which has a calculated pKa of 4.24. As the chain length and aliphatic substitution of a spacer chain increase, generally the pKa increases to a maximum of about 4.5. However, as the chain becomes unsaturated, the pKa decreases, as seen with fumaric (pKa 3.13) and maleic (pKa 2.39) acids. This is also illustrated in the difference in chains of the same length and increasing unsaturation, in 6-carbon chains from adipic acid (pKa 4.39, # unsat=0) to dihydromuconic acid (pKa 3.96, # unsat=1) to muconic acid (pKa 3.77, # unsat=2). Adding electronegative groups (such as O, S, or C(O) to a chain of the same length also decreases the pKa. This is illustrated in 5-member chains, going from the saturated, unsubstituted glutaric acid (pKa 4.33; C—C—C—C—C) to thiodiglycolic acid (pKa 3.25; C—C—S—C—C) to diglycolic (pKa 2.73; C—C—O—C—C) to 3-ketoglutaric (pKa 2.49; C—C—C(O)—C—C). Changing the straight chain to an aromatic one also decreases the pKa, from cyclohexanedioic acid (pKa 4.51; saturated 6-member ring) to 4-carboxybenzoic acid (pKa 3.49; aromatic 6-member ring). Furthermore, adding an electronegative heteroatom to the aromatic ring further decreases the pKa, as illustrated by comparing norcamphoric acid (pKa 4.23; 5 member ring) to 2,5-tetrahydrofuran-dicarboxylic acid (pKa 3.04; 5-member ring, with O substitution) and to furandicarboxylic acid (pKa 2.28; 5-member aromatic ring with O subst.). Data presented herein indicates that a pKa<4.5, 4.4, 4.3, 4.2, 4.1, or 4.0 may be preferred.

Other considerations for design include that the presence of multiple ionizable groups may be unfavorable, as would be restriction around the free (unconjugated) hydrophilic group.

Conversely, additional bulk surrounding the conjugated acid (2,2,6,6 tetramethylpimelic acid) would be useful for extending the stability of the conjugate if needed, but might also inhibit the interaction with lipid transporters. In this case, assymetrical dicarboxylates (2,2 dimethylpimelic acid) might be used instead. Those of skill in the art are familiar with calculating, for example, pKa values, and with methods of testing prodrugs as described herein, and would be capable of taking these parameters into account when practicing the invention, without undue experimentation.

### Therapeutic Agents

The drug or molecule of interest that forms part of the prodrug is generally a large (e.g. molecular weight greater than about 400), lipophilic therapeutic agent with calculated LogP values of 1.5 or greater. However, this need not always be the case. Any agent which can be advantageously delivered in the form of a “prodrug” as described herein may be attached to a lipid-like moiety that is a substrate for uptake by a fatty acid transport mechanism and administered as described herein.

Further, the lipophilic drug moiety is not a dipeptide compound which is an α-aminocarboxamide containing a 3-amino-2-hydroxy-4-substituted-phenylbutanoyl with a five membered ring connected via an amide bond, or a derivative thereof, as described and depicted as Formulas I and II in U.S. Pat. No. 6,673,772 to Mimoto et al., the complete contents of which is herein incorporated by reference. However, prodrugs comprising this entity may be administered using the methods of the invention.

Those of skill in the art will recognize that LogP values of compounds can be readily obtained, e.g. using computer programs such as that which is available at the website located at scifinder.cas.org/scifinder, which also provides a method for calculating pKa values.

The therapeutic compound comprises at least one chemically reactive functional group, for example, a hydroxyl or an amine, which can be conjugated by means of e.g. esterification or amidation, to the transport moiety. The functional group may be aliphatic (saturated or unsaturated carbon), or aromatic. If the functional group is β-unsaturated (as in tipranavir), keto-enol tautomerism may exist. This does not preclude conjugation, but may require altered reaction conditions (stronger base, higher temperature, longer reaction time) to obtain higher yields of the prodrug. If the group is aromatic, the transport moiety should have either a higher pKa or have additional bulk to protect the linkage, due to decreased chemical stability caused by aromaticity. Generally, aliphatic alcohols (e.g. hydroxyls) are preferred as functional groups.

Exemplary therapeutic compounds that may be derivatized by the addition of a lipid or lipid like moiety (lipophilic drug moieties) as described herein include but are not limited to: various protease inhibitors or other agents which are used to treat HIV or other diseases, such as lopinavir, ritonavir, saquinavir, nelfinavir, atazanavir, indinavir, tipranavir, darunavir, amprenavir, ziagen (abacavir sulfate, as described in U.S. Pat. No. 5,034,394), epzicom (ahbacavir sulfate/lamivudine, as described in U.S. Pat. No. 5,034,394), hepsera (adefovir dipivoxil, as described in U.S. Pat. No. 4,724,233), agenerase (amprenavir, as described in U.S. Pat. No. 5,646,180), reyataz (atazanavir sulfate, as described in U.S. Pat. No. 5,849,911), rescriptor (delavirdine mesilate, as described in U.S. Pat. No. 5,563,142), hivid (dideoxycytidine; zalcitabine, as described in U.S. Pat. No. 5,028,595), and videx (dideoxyinosine; didanosine, as described in U.S. Pat. No. 4,861,759); HIV protease inhibitors as described in US patent application 2007/0066664, as well as antiviral compounds such as those described in US patent application 2010/0022508; etc.; various anti-cancer drugs for brain and other drug-resistant cancers, including temozolomide; tyrosine kinase inhibitors such as sorafenib, erlotinib, gefitinib, imatinib, and pazopanib; rapamycin (sirolimus); steroidal compounds including estrogens, progestins, androgens, and corticosteroids such as estradiol, 2-methoxyestradiol, ethynylestradiol, testosterone, cortisol, mestranol, hydroxyprogesterone, medroxyprogesterone, etc.; substances that are used to treat or prevent cancer or other conditions such as raloxifene, lasofoxifene, basedoxifene, resveratrol, curcumin, etoposide, capmtothecin, CPT-11, topotecan, irinotecan, extecan, lurtotecan, DB67, BNP1350, ST1481, CKD602, and other analogues of camptothecein, paclitaxel (“taxol”, docetaxel, and other anticancer taxanes, vincrisitine, vinblastine, fingolimod, raltegravir, elvitegravir, MK-2048, lersivirine, daunorubicin (daunomycin), doxorubicin, epirubicin, idarubicin, and other anti-cancer anthracyclines, etc. Each of the patents and applications referenced herein being incorporated by reference. Furthermore, the invention includes modifications (derivatives) of each of the above listed drugs which conceal hydrophilic groups, such as —OH and —NH—) by forming hydrolyzable ester or amide bonds. These modifications of the lipophilic drug moiety serve to make the liphophilic drug moiety effectively more lipophilic.

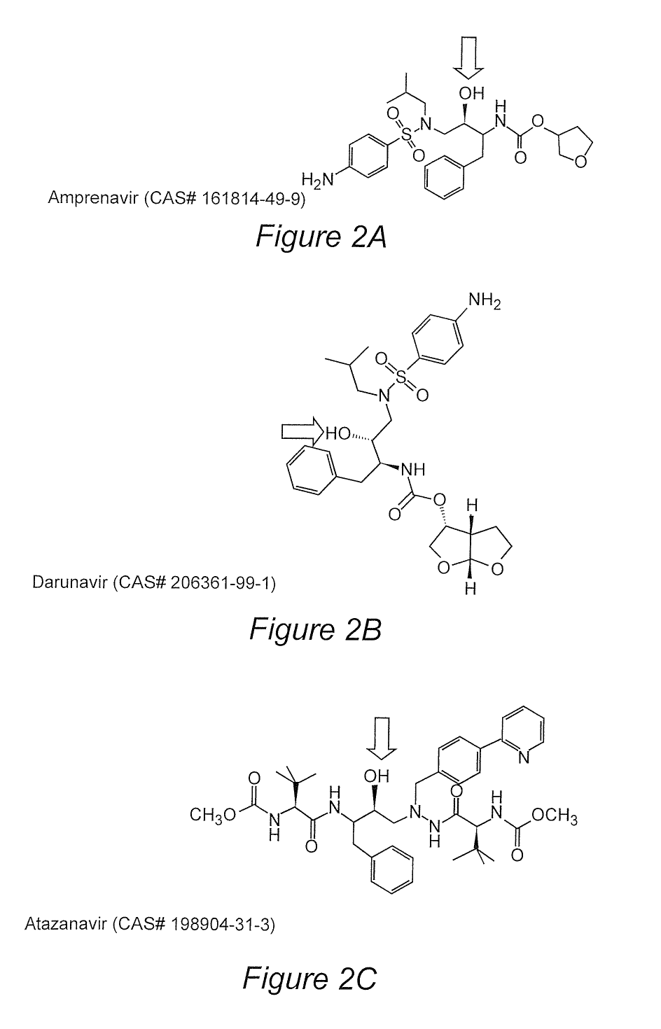

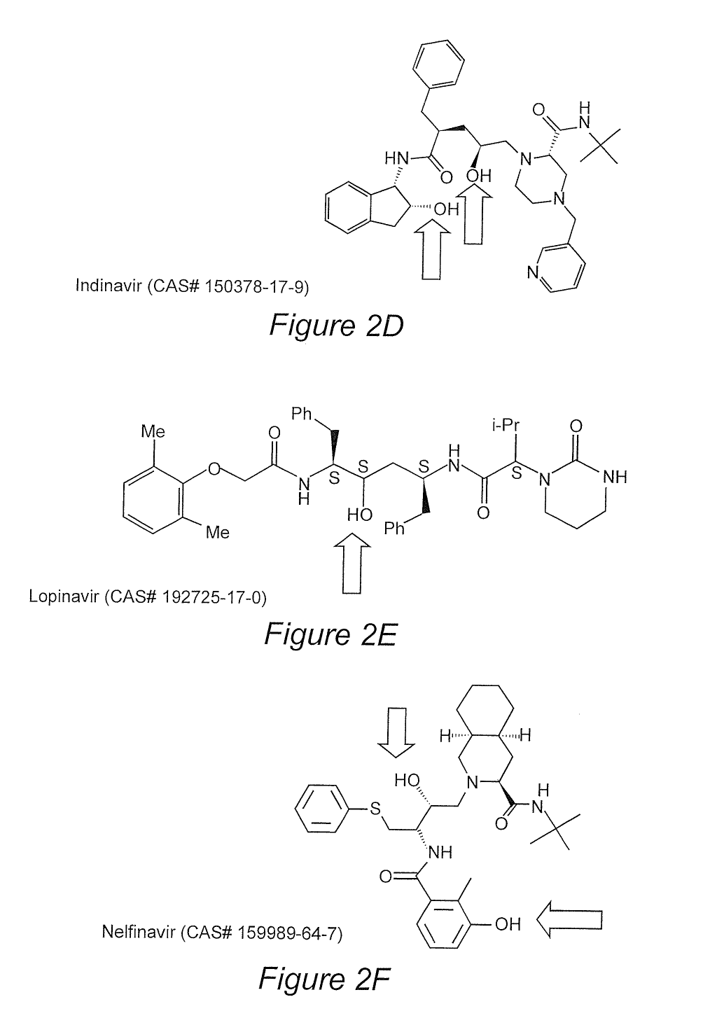

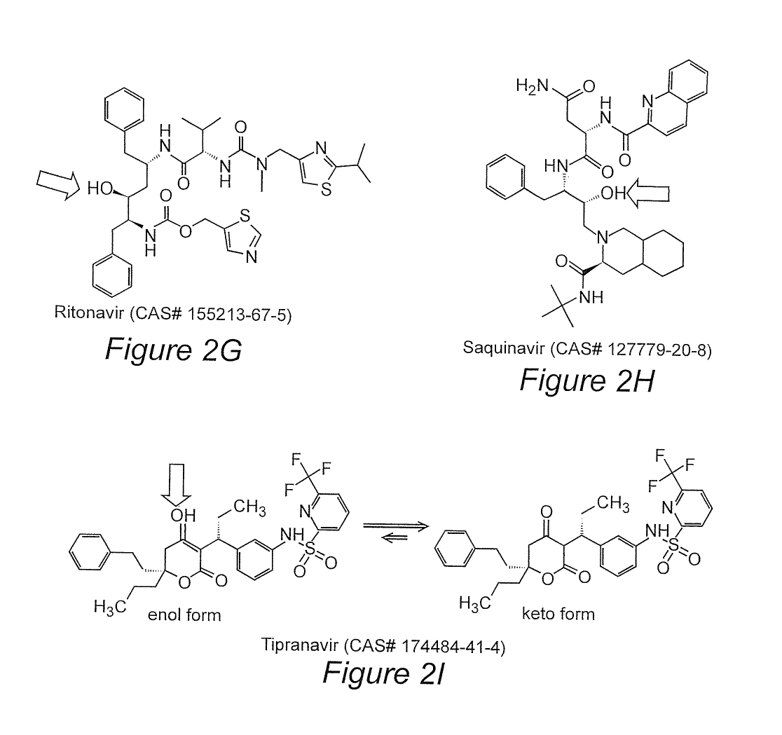

Exemplary HIV protease inhibitors that may be delivered in this manner are depicted in FIGS. 2A-I, which also indicates the reactive group (OH) that serves as a point of attachment between the inhibitor and the lipid-like moiety. In some embodiments, the compounds are lopinavir, ritonavir, saquinavir, nelfinavir, atazanavir, indinavir, tipranavir, darunavir, amprenavir, fosamprenavir, brecanavir (CTP-518 [GW640385], a novel HIV protease inhibitor developed by replacing certain key hydrogen atoms of atazanavir with deuterium), mozenavir (DMP450), TMC310911 by Tibotec Therapeutics, L-756,423 by Merck, Mozenavir (DMP450) by Triangle Pharmaceuticals, PPL-100 (MK8122) by Ambrilla/Procyon Biopharma, RO033-4649 by Roche, SP1256 by Sequoia Pharmaceuticals, as well as other HIV protease inhibitors that are currently under development.

Furthermore, improved uptake activity for the prodrug can be achieved when the transport moiety changes the three dimensional structure of the unmodified portion of the parent compound.

### Linkage Between the Transport Moiety and the Therapeutic Agent

Generally the chemical bonds which join (attach, link, etc.) the lipid-like moiety to the drug or molecule of interest are hydrolyzable via enzymatic hydrolysis, e.g. by esterases, phosphatases, amidases, etc. that occur in cells and tissues of the body. However, in cases, non-enzymatic hydrolysis may also occur, e.g. adventitiously, or the linkage may be designed to be susceptible to cleavage under physiological conditions (e.g. via an intermolecular cyclization-elimination reaction via imide formation, etc.). Exemplary bonds include but are not limited to, for example: ester, phosphoester, amide, carbonate bonds,

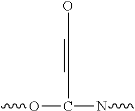

etc. The bonds are generally, although not always (see below), hydrolyzable under physiological conditions. By “physiological conditions” we mean that the bonds are cleavable by non-enzymatic hydrolysis at a pH of from about 6.5 to about 7.5, in an aqueous milieu. Preferably, cleavage does not occur immediately after administration, but after uptake by the fatty acid transporter. Thus, the half-life of the intact prodrug is generally in the range of from about 1 minute to about 5 hours, and usually from about 5 minutes to about 4 hours, or from about 10 minutes to about 3 hours, or even from about 20 minutes to about 2 hours. Methods of adjusting designing a prodrug by varying components in order to modulate the half life are discussed above.

While the transport moiety is generally linked to the drug or molecule of interest via a chemical bond that is hydrolyzable under physiological condition, this need not always be the case. In some embodiments, the bond between the active agent and the lipid-like moiety is not hydrolyzable but the active agent retains sufficient activity to have a beneficial effect when administered as described herein. Such non-hydrolyzable prodrugs are also encompassed by the present invention in that they are still taken up by fatty acid transport systems, and thus still delivered to tissues and cells which are accessible via these systems. In this embodiment, the therapeutic agent retains at least about 25, 30, 35, 40, 45, 50, 55, 60 65, 70, 75, 80, 85, 90, 95 or even 100% of its activity, even when conjugated (attached) to the lipid-like moiety. Exemplary non-hydrolyzable bonds include but are not limited to amides and esters of acids with pKa values>about 4.

### Exemplary Prodrugs

The prodrugs of the invention include any prodrug formed as described herein, by combining any drug or substance described herein, together with any transport moiety described herein, as well as various geometric isomers thereof (e.g. R and S isomers). In addition, radioactive forms of the prodrugs (e.g. ³H, ¹⁴C, etc.) and deuterated forms (in which one or more H atoms is replaced by deuterium) are encompassed.

Exemplary prodrugs of the invention include but are not limited to: mono-esters and diglycolic esters of various drugs such as lopinavir, ritonavir, saquinavir, nelfinavir, atazanavir, indinavir, tipranavir, estradiol, methoxyestradiol, resveratrol, etc. In particular, lopinavir prodrugs include succinyl-lopinavir, diglycolic-lopinavir, thiodiglycolic-lopinavir, funaryl-lopinavir, muconyl-lopinavir, adipoyl-lopinavir, thiopropionyl-lopinavir, 2-ketoglutaryl-lopinavir, and 3-ketoglutaryl-lopinavir, as depicted in FIGS. 3A-I, each of which has been synthesized as described herein. Other lopinavir prodrugs that may be synthesized in a similar manner include but are not limited to cyclohexanedioyl-lopinavir, glycerosuccinyl-lopinavir, various lopinavir carbamates, citrosucciinyl-lopinavir and malosuccinyl-lopinavir, as depicted in FIG. 3J-N.

Exemplary mono-esters of other therapeutic agents include but are not limited to: diglycolic-ritonavir, diglycolic-saquinavir, diglycolic-nelfinavir, diglycolic-atazanavir, diglycolic-indinavir, diglycolic-tipranavir, diglycolic-2 methoxyestradiol, diglycolic-estradiol, and succinic-resveratrol, as depicted in FIGS. 3 O to Y.

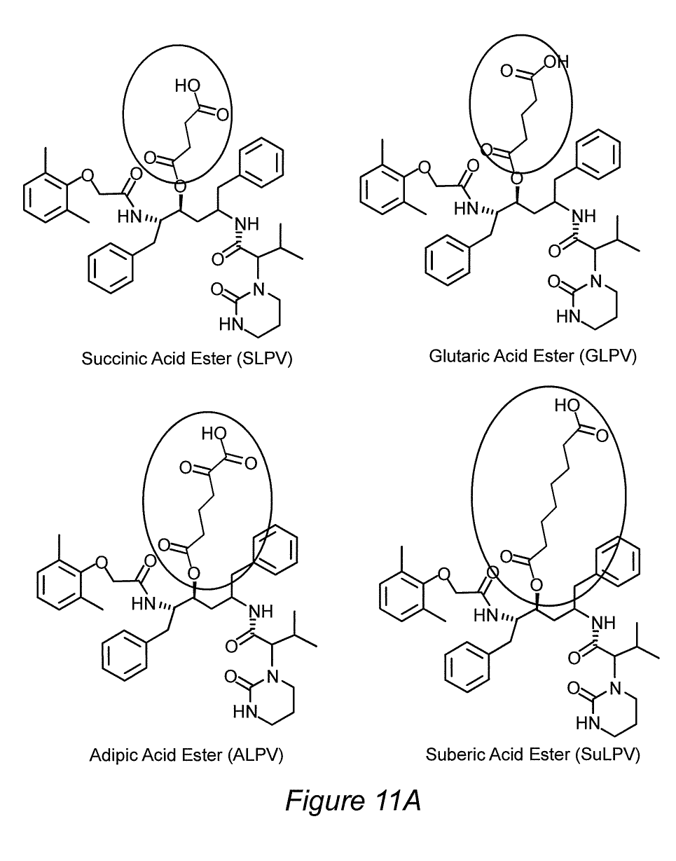

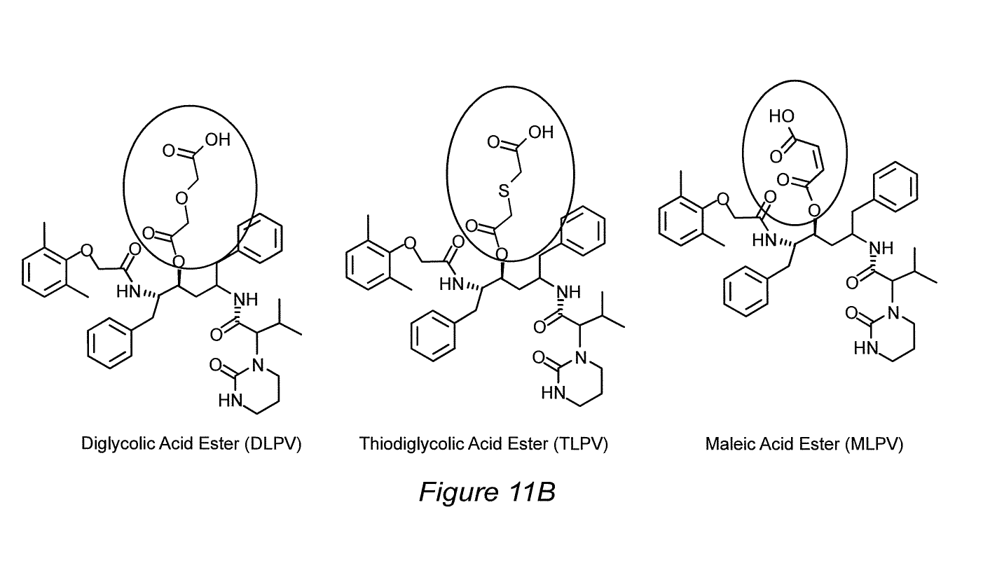

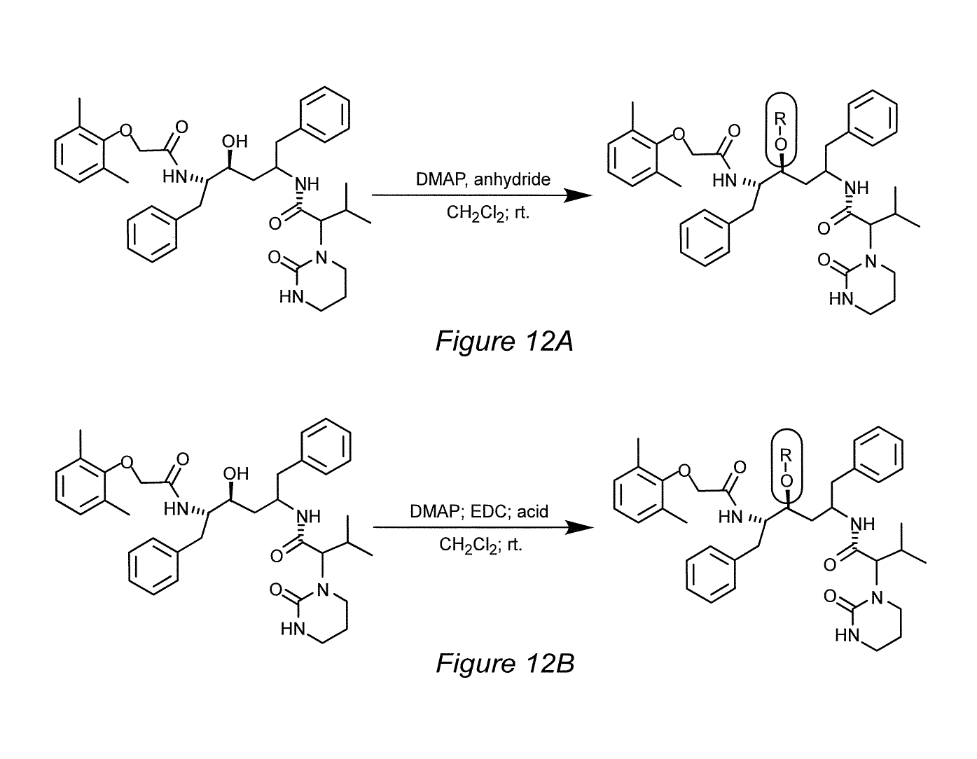

FIGS. 11a-b show examples seven different prodrugs according to the present invention which can be prepared according to the general synthesis routes shown in FIGS. 12a-b.

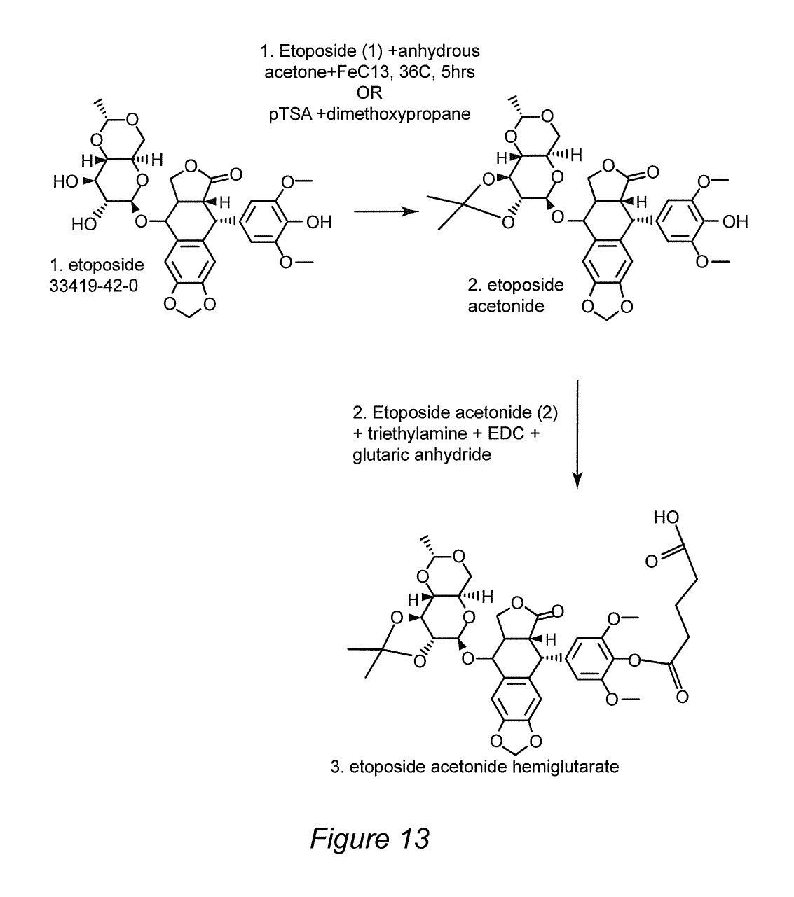

FIG. 13 shows an exemplary synthesis procedure for preparing the prodrug 3, etoposide acetonide hemiglutarate. In this embodiment, a hydrophilic drug (etoposide) is made hydrophobic by concealing its hydrophilic functional groups, and the modified drug is attached to the transport moiety.

### Compositions

The invention also provides pharmaceutical compositions for administration to patients in need thereof. The compositions include one or more substantially purified prodrugs as described herein, and a pharmacologically suitable (physiologically compatible) carrier. The preparation of such compositions for administration to living patients is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of prodrug in the formulations may vary. However, in general, the amount in the formulations will be from about 1-99%.

The compositions of the invention may contain or be administered with other beneficial substances, e.g. nutritional substances, appetite stimulants, substances that stimulate the immune system, antibiotics, other antiviral agents (e.g. ritonavir), for example, in a “cocktail”, etc.

### Methods of Using the Prodrugs

The invention also provides methods for treating a condition or disease in a patient in need thereof. In some embodiments, the patient is suffering from a disease or condition wherein the patient is immunocompromised and suffers from infection by a disease agent such as a virus, bacteria, protozoa, etc. Exemplary patients include but are not limited to patients infected with HIV. Other types of patients may also be treated, e.g. those for who administration of a steroid would be beneficial. Any patient who might benefit from administration of a prodrug as described herein may be treated by the methods of the invention. The methods involve administering to the patient at least one prodrug as described herein. The prodrug compositions (preparations) of the present invention may be administered by any of the many suitable means which are well known to those of skill in the art, including but not limited to by injection, inhalation, orally, intravaginally, intranasally, topically, as eye drops, via sprays, etc. In preferred embodiments, the mode of administration is orally or by injection. In addition, the compositions may be administered in conjunction with other treatment modalities such as substances that boost the immune system, various chemotherapeutic agents, antibiotic agents, and the like.

The quantity of prodrug that is administered varies according to several factors, including but not limited to: the identity of the drug or molecule of interest, the identity of the lipid-like moiety, the rate of hydrolysis of the prodrug in vivo, the rate of uptake of the prodrug by a fatty acid transporter mechanism, the physical characteristics of the patient that is being treated (e.g. gender, age, overall health, stage of disease, response to drugs, etc.); the particular disease or condition that is being treated; the type of tissues or cells that are targeted; and other factors. The quantity of prodrug is best determined by a skilled medical practitioner such as a physician, and guidelines are typically worked out e.g. in animal and clinical trials. Generally, the objective is to achieve a local concentration of prodrug and/or released drug at the site of action of from about 1 nM to about 10 depending on the pharmacology of the active compound.

The compositions and methods of the invention are generally used to treat mammals, e.g. humans, but veterinary uses are also contemplated.

Diseases and Conditions that can be Treated Using the Prodrugs of the Invention

A plethora of disease and conditions can be treated using the compositions and methods of the invention, including but not limited to: HIV; various cancers such as brain cancer, colon cancer, choriocarcinoma, hepatocarcinoma, leukemia, renal cancer, lung cancer; various disorders of the central nervous system (CNS) such as HIV encephalopathy, Alzheimer's disease, Parkinson's disease, epilepsy and seizure disorders, and various other neuropathologies; psychiatric illnesses such as depression, bipolar disorder, anxiety and others; addictions such as dependence upon opiates, alcohol, stimulants, and hallucinogens; various fetal disorders which can be treated by transplacental delivery of therapeutic agents such as HIV prophylaxis and infection; and cardiac arrhythmias and abnormalities, etc. Any disease or condition that is amenable to treatment, amelioration, or prevention by the delivery of therapeutic agents via a fatty acid transport mechanism may be treated by the compositions and methods described herein. In some embodiments, any disease or condition that is amenable to treatment, amelioration, or prevention via administration of a large, lipophilic drug as described herein may be treated by the compositions and methods of the invention.

In one embodiment, the compounds and methods of the invention are used to treat multidrug resistance in patients such as cancer patients. In other words, the technology can also be applied to the delivery of drugs for the treatment of multidrug resistant tumors (cancers) or seizure foci. Many of the drugs used to treat cancer and seizure disorders are substrates for multidrug resistance transporters such as P-glycoprotein (MDR1; gene symbol ABCB 1), Breast Cancer Resistance Protein (BCRP; gene symbol ABCG2), as well as some of the Multidrug Resistance-associated Proteins (MRP's; gene symbols ABCC1 through ABCC9) as well as the Ral-binding protein RLIP76 (gene symbol RALBP1). These are proteins which pump the drugs out of the target cells or tissues, thus preventing their therapeutic benefits. The present invention would circumvent these transporters, carrying the prodrugs across the membrane into the cells or tissues, where the active compound would be released.

All articles, patents, and patent applications cited herein are hereby incorporated by reference in entirety.

### EXAMPLES

### Background

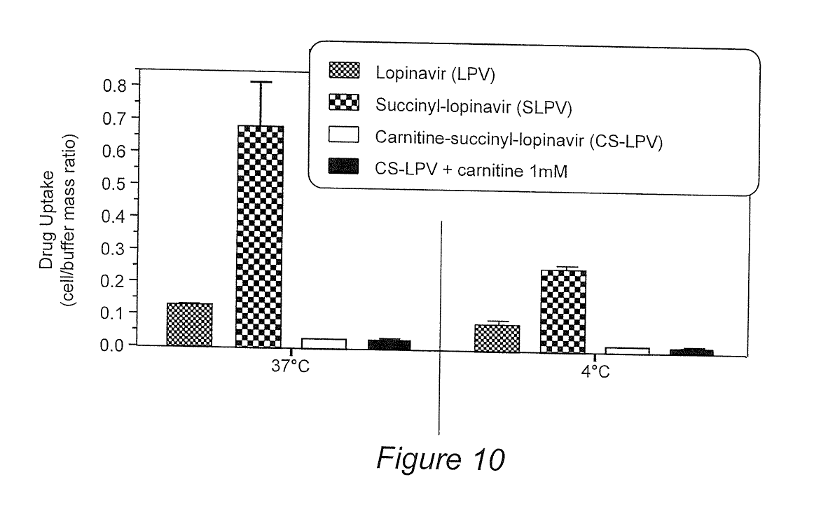

Succinyl-lopinavir (SLPV) was originally synthesized as a synthetic intermediate, the original goal being to attach other types of nutrients (e.g carnitine) to the SLPV, in an attempt to imprive their cellular uptake. Carnitine-succinyl-lopinavir (CS-LPV) was thus synthesized and its uptake into BeWo cells was compared to the uptake for the starting material, LPV and the intermediate, SLPV. The results are depicted in FIG. 10. Disappointingly, CS-LPV uptake was very low, worse than that of LPV, so this was not an improvement. Surprisingly though, SLPV uptake was much greater than that of LPV. So a new set of investigations undertaken. Temperature dependence was investigated and it was confirmed that non-specific binding was not responsible. Rather, a biological transporter was being utilized. Several logical candidates transporter systems were tested by using broad-based inhibitors, but little or no effect was observed even at high concentrations that should have abolished the activities of OATs, OATPs, and MCT systems. A literature search suggested that fatty acid transporters should be considered. Upon testing, we observed that linoleic acid inhibited uptake of SLPV but not LPV. Thus, it is hypothesized that FATP4, which is expressed in the placenta and the brain, may the transport system by which the prodrugs of the invention are taken up.

The following Examples provide further details of how to make and use the invention.

### Example 1

### Synthesis of Prodrugs

### I. Generic Synthesis Scheme

Generic reaction description: Drug (R) is dissolved in a suitable anhydrous organic solvent (such as dimethylformamide, dichloromethane, acetonitrile, dimethylsulfoxide) in the presence of an organic base (such as pyridine, dimethylaminopyridine, triethylamine) with 4A molecular sieves. To perform esterification or amidation, several possibilities may occur, depending upon the availability of starting materials, as below.

A. Anhydrides: If the desired acid anhydride is available, this is generally preferred since it provides cleaner and more efficient reactions. The acid anhydride is either added directly to the reaction mixture above, or dissolved in a suitable organic solvent. After the addition of the acid anhydride, the reaction is allowed to proceed under inert atmosphere, typically at 20-80° C. for 2-14 hours, while protected from light.

B. Free Acids: If only the free acid is available, then a catalyst such as dicyclohexylcarbodiimide (DCC) or N-(3-Dimethylaminopropyl)-N′-ethylcarbodiimide hydrochloride (EDC.HCl) should be used in a molar ratio of >1 with the free acid. The free acid is either added directly to the reaction mixture above, or dissolved in a suitable organic solvent. After the addition of the acid, the reaction is allowed to proceed under inert atmosphere, typically at 20-80° C. for 2-14 hours, while protected from light.

C. Acid Chlorides: Generally the use of acid chlorides is not preferred, due their high reactivity and formation of side products. However, the use of acid chlorides may be required for some slow or hindered reactions. The acid chloride should be added slowly and gradually with stirring, under inert atmosphere. After the addition of the acid chloride, the reaction is allowed to proceed under inert atmosphere, typically at 20-80° C. for 2-14 hours, while protected from light.

Workup: The reaction is filtered, the solvent is evaporated, and the products are separated on silica gel (dichloromethane/methanol) or by reversed-phase HPLC (C18; mobile phase containing methanol, acetonitrile, and aqueous ammonium acetate) with detection by UV absorbance.

### II. Synthesis of Succinyl-Lopinavir

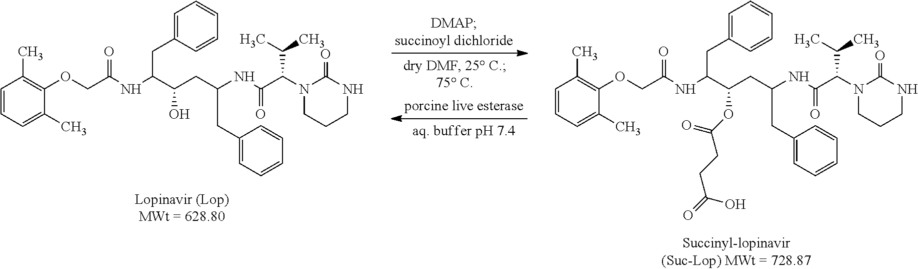

### Chemical Synthesis

Succinyl-lopinavir (SLPV) was synthesized from lopinavir powder (BetaPharma, Inc) by dissolving lopinavir (100 μmol) and N,N-dimethyl-4-aminopyridine (DMAP; 130 μmol) in anhydrous dimethylformamide (DMF). To this solution, succinoyl dichloride (SucCl₂; 150 μmol) was added, diluted 1:10 in anhydrous DMF, under inert atmosphere over 4A molecular sieves in a total reaction volume of 1 ml. Reactions proceeded with warming (75° C.) and monitoring by reversed-phase HPLC with gradient elution and detection by UV (254 nm) and fluorescence (ex 266 nm, em 300 nm). Upon completion, the reaction was evaporated under reduced pressure, reconstituted with methanol/aqueous acetic acid, and purified by preparative-scale reversed-phase (C-18, 4 g) flash chromatography (Agela, Inc) with reduced-pressure evaporation of eluted fractions to achieve>95% chemical purity. 3H-SLPV was synthesized from 3H-lopinavir (Moravek, Inc.) by drying 100 μCi (typically ˜1 μmol) and adding DMAP (1.3 μmol) and SucCl2 (1.5 μmol in 1 ml DMF as above. The reaction product was worked up as above, but with purification on an Alltima HP C18 4.6×100 mm 3 μm column, with evaporation of eluted fractions to achieve radiochemical purity>97%.

Chemical Identification: To determine the chemical identity of SLPV, ester compounds were incubated with porcine liver esterase (50 U/ml) at 37° C. in borate/phosphate buffer at pH 8.0. Reaction samples were terminated with 2 volumes of methanol/acetic acid (1%) and analyzed by HPLC. The loss of the peak area for the lopinavir ester was recovered in the gain of lopinavir peak area, indicating hydrolysis to release the parent compound. Next, compounds were dissolved in 80% acetonitrile/20% aqueous (0.1% trifluoroacetic acid) and infused onto a Waters Micromass ZMD mass spectrometer, with atmospheric pressure chemical ionization in positive ion mode, and monitoring from 300 to 800 m/z. Spectra of products was compared to solvent alone or lopinavir to determine the molecular ion. Finally, to confirm the structure of lopinavir esters, compounds were reconstituted with CDCl₃ for NMR analysis. The identity of lopinavir esters was confirmed by proton NMR spectrometry using the Varian Mercury 300-MHz spectrometer. ³H-SLPV is identified by HPLC retention time using SLPV as an authentic standard.

### III. Synthesis of Diglycolic-Lopinavir

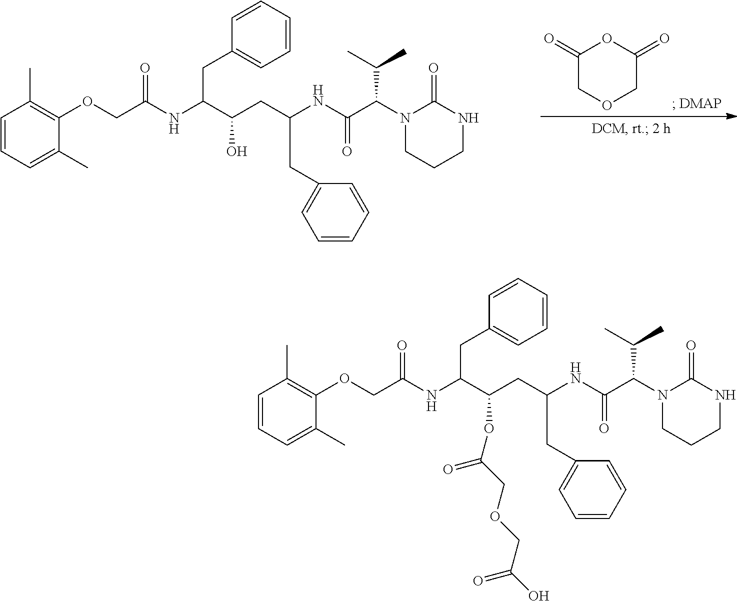

### Reaction:

50 mg, 0.08 mmol Lopinavir
36 mg, 0.32 mmol DMAP
28 mg, 0.24 mmol Diglycolic anhydride
Mixed together, 2 mL of CH₂Cl₂ was added and stirred for 2 hours (no Lopinavir left) Workup: dry under reduced pressure; dissolve with diethyl ether and using 1 M HCl wash twice, saturated NaHCO₃ solution wash once, dry overnight by Na₂SO₄. After that, centrifuge to dry them. Using a little CH₂Cl₂ to dissolve and then separate product by column chromatography on silica using CH₂Cl₂:MeOH as follows: 50:1 (50 ml), 25:1 (50 ml) and then 15:1 (20 ml) to elute the product.

### Separation:

1. Silica gel: Using CH₂Cl₂:MeOH 15:1, R_{f} is 0.3. I₂ was used as reagent to color the product
2. HPLC (Microsorb-MV 4.6×100 mm 3μ C18 from Agilent): UV 220 nm. Flow rate: 1 mL/min. A: ACN; B: MeOH (50%)+50 mM, pH 5.5 NH₄AC (50%); Mobile phase: from 0-10 min, 40% A-55% A. Retention time: 4.0 min

NMR (CDCl₃): 7.11-7.24 (12H, m), 6.91-7.00 (4H, m), 6.72 (1H, s), 5.84 (1H, s), 5.14 (1H, m), 4.82 (1H, m), 4.37 (1H, m), 4.1-4.27 (8H, m), 3.01-3.19 (3H, m), 2.80-2.95 (3H, m), 2.61-2.75 (2H, m), 2.07-2.23 (7H, m), 1.90-1.98 (1H, m), 1.67-1.74 (2H, m), 1.49-1.52 (1H, m), 0.86 (3H, d), 0.82 (3H, d).

ES⁻: m/z: 743.61 (peak: M−H)

### IV. Synthesis of Thiodiglycolic-Lopinavir

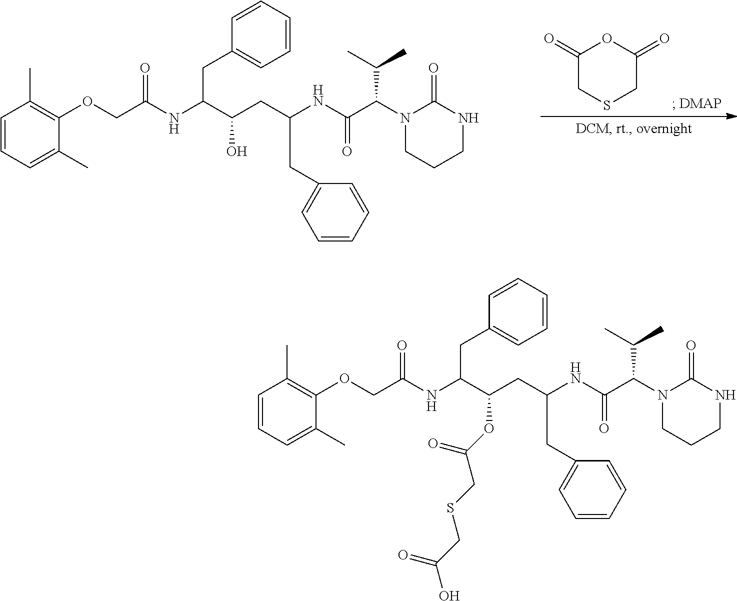

### Reaction:

50 mg, 0.08 mmol Lopinavir
36 mg, 0.32 mmol DMAP
32 mg, 0.24 mmol Thiodiglycolic anhydride
Mixed together, 2 mL of CH₂Cl₂ was added and stirred overnight
Workup: dry under reduced pressure; dissolve with diethyl ether and using 1 M HCl wash twice, saturated NaHCO₃ solution wash once, dry overnight by Na₂SO₄. After that, centrifuge to dry them. Using a little CH₂Cl₂ to dissolve and then separate product by column chromatography on silica using CH₂Cl₂:MeOH as follows: 50:1 (50 ml), 25:1 (50 ml) and then 15:1 (20 ml) to elute the product.

### Separation:

Using CH₂Cl₂:MeOH 30:1 50 mL, and then 15:1 get a mixed product. After that, using silica gel plate CH₂Cl₂:MeOH 15:1 to separate the product. R_{f} is 0.35

NMR (CDCl₃): 7.18-7.31 (12H, m), 6.93-7.01 (4H, m), 6.63 (1H, d), 6.37 (1H, s), 4.99-5.08 (2H, m), 4.48 (1H, m), 4.08-4.26 (3H, m), 3.33-3.49 (6H, m), 2.73-3.25 (7H, m), 2.52 (1H, m), 2.15 (6H, s), 1.84 (1H, m), 1.64 (2H, m), 1.39 (1H, m), 1.26 (1H, s), 0.87 (3H, d), 0.80 (3H, d)

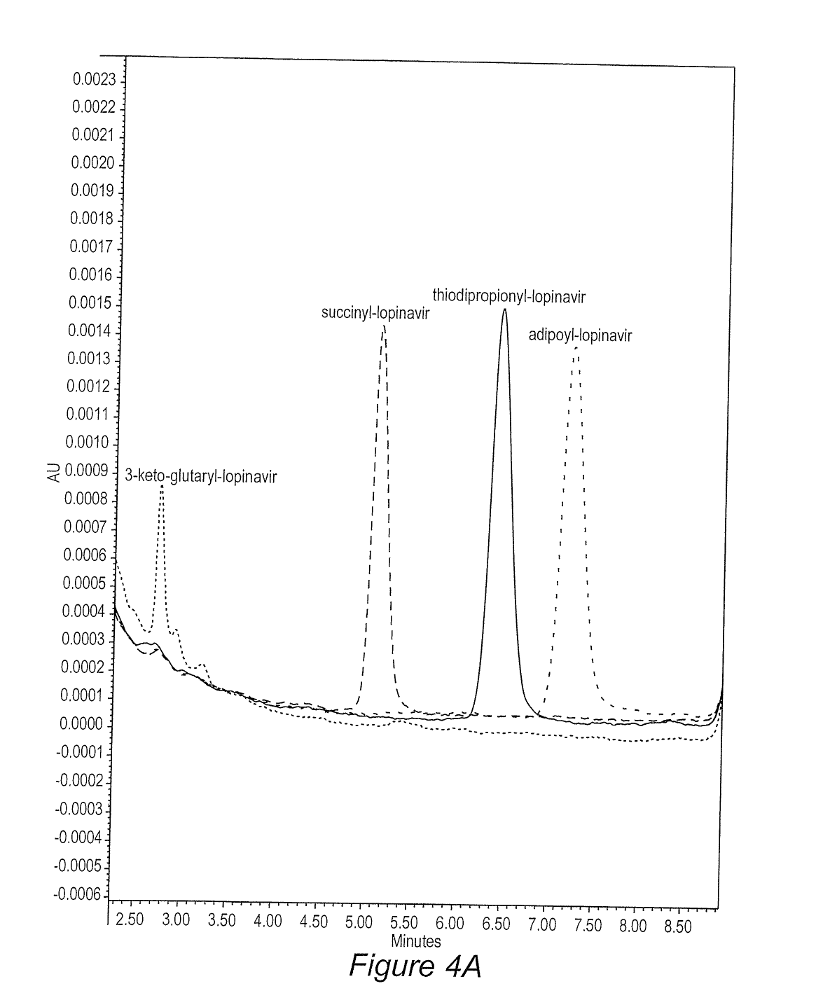

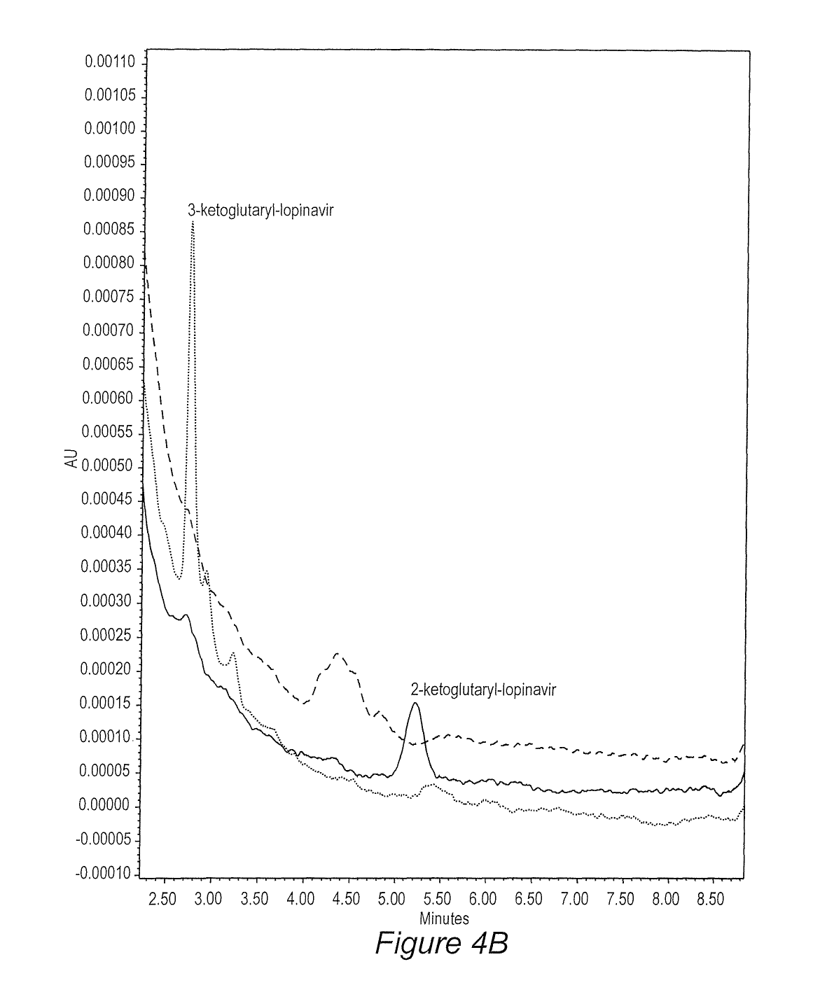

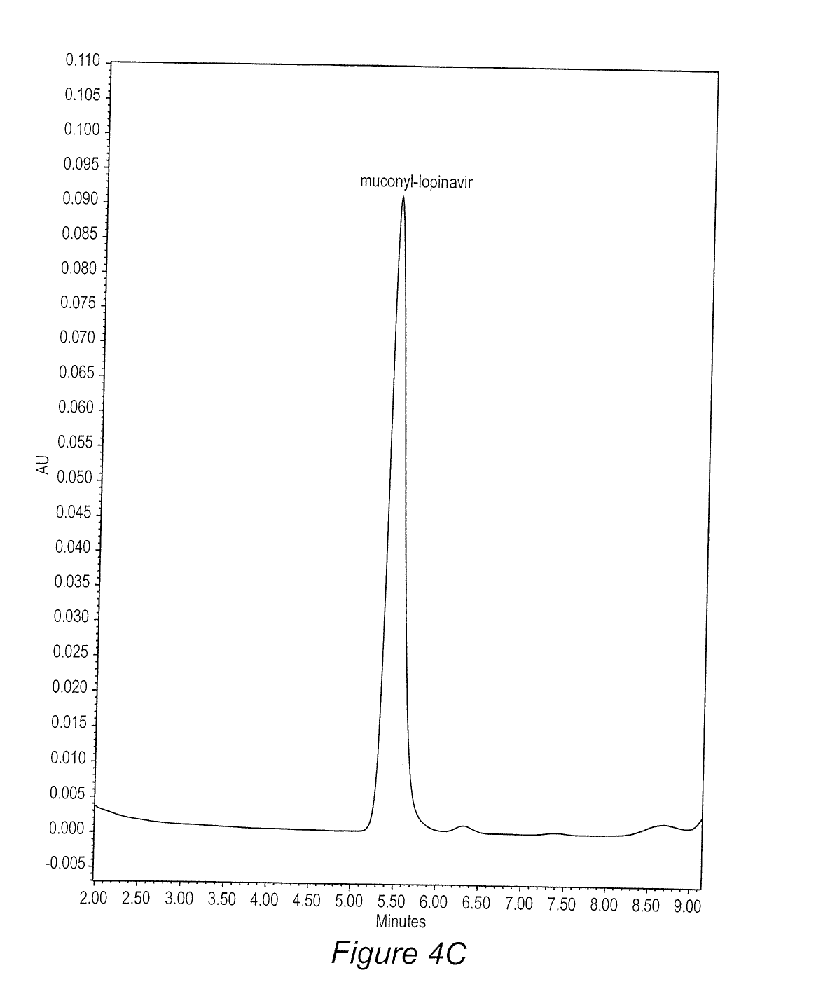

Exemplary HPLC results for representative compounds are shown in FIGS. 4A-C.

### Example 2

### Elucidation of Uptake Mechanism for Succinylated Lopinavir

Experiments were conducted to differentiate between the uptake mechanisms of lopinavir (LPV) and succinylated lopinavir (SLPV). As shown in the Table 1 below, several inhibitors of putative uptake transport mechanisms for SLPV had no effect. Specifically, bromosulfophthalein (BSP) is an inhibitor of several organic anion transporters (OATs; gene family SLC22A), as well as organic anion transporting polypeptides (OATPs; gene family SLCO). Even at 250 μM, BSP had no effect on LPVor SLPV uptake. Also, valproic acid and monoethylsuccinate as inhibitors of monocarboxylate transporters (MCTs; gene family SLC16A) showed negligible inhibition of SLPV uptake at 1 mM. Probenecid also inhibits many transporters, including OATs, but did not impact SLPV uptake. Finally, taurocholate is a classic substrate for bile salt transporters, including the sodium-dependent taurocholate transporter (NTCP; SLC10A1), the apical bile salt transporter (ASBT; SLC10A2), and certain OATPs. At a typical concentration of 100 μM, taurocholate had no impact on SLPV uptake. These data are consistent with the lack of involvement of OATs, OATPs, OCTNs, MCTs, and bile salt transporters in SLPV uptake in BeWo cells.

SLPV is a large (MWt=728.87) lipophilic molecule with a free carboxylic acid group that should be a substrate for fatty acid transporters. Linoleic acid has previously been demonstrated to be preferentially transported in the apical-to-basolateral direction (analogous to the maternal-to-fetal direction across the placenta).³⁸ Significantly reduced uptake of SLPV was observed in the presence of 40 μM linoleic acid (LA), whereas LPVuptake was unaffected.

### Example 3

### Targeting Anti-Retroviral Drugs to Pharmacologic Sanctuaries Using Placental Delivery

Human Immunodeficiency Virus (HIV)—infected patients are defined as having the Acquired Immunodeficiency Syndrome (AIDS) if they have CD4 cell count of less than 200/mm3. Since the onset of the epidemic, close to 60 million people have been infected with the virus, with almost 20 million dying from its complications. Of the more than 40 million people with HIV infection today, close to half of them are women, and more than 3 million are children under the age of 15.[1] To treat HIV infected patients, multiple antiretroviral drugs are used in what is known as Highly Active Antiretroviral Therapy (HAART).

Amongst the anti-HIV drugs, nucleoside analogs, nucleoside reverse transcriptase inhibitors, and non-nucleoside reverse transcriptase inhibitors achieve cord plasma to maternal plasma concentration ratios approaching unity. However, the HIV protease inhibitors do not appear to cross the placenta nearly as readily, with cord plasma concentrations reaching only=30% of maternal plasma concentrations, decreasing their efficacy. ATP-binding cassette (ABC) transporters in the apical syncytiotrophoblast membrane are important in limiting fetal penetration of protease inhibitors, as demonstrated in studies in the placenta and other tissues.[2]

In pregnant HIV infected patients, however, HAART serves two goals, providing adequate treatment for the mother and preventing viral transmission to the fetus. The use of HAART regimens has led to a significant reduction in occurrence of perinatal transmission to less than 2%.[3] According to 2010 guidelines, lopinavir/ritonavir is the only recommended agent in the category of protease inhibitors for use in HIV-infected pregnant patients.

HIV protease inhibitors, as substrates of ABC transporters, have been the targets of many studies that have been carried out to see the effect of pharmacokinetic changes during pregnancy, on the placental transfer of this category of drugs. The literature shows several cases in which efflux transporters are responsible for low drug concentrations reaching the fetus. For example, a deficiency in mouse placental P-glycoprotein has been shown to enhance fetal susceptibility to chemically induced birth defects by avermectins.[4] Similarly, Smit et al. showed that 2.4-, 7-, or 16-fold more [³H] digoxin, [¹⁴C] saquinavir, or paclitaxel, respectively, entered the P-glycoprotein deficient Abcb1a−/−/1b−/− fetuses than entered wild type fetuses in the study carried out in mice.[5] They also used the P-glycoprotein inhibitors PSC833 or GG918 to show that blocking P-glycoprotein using these inhibitors resulted in increased transplacental passage of these drugs into the fetus. Molsa et al. observed similar results in studies carried out with human placentae, in which preperfusion with PSC833 increased the maternal-to-fetal transfer of saquinavir by 7.9-fold (0.18%±0.09% vs 1.4%±0.67%), and preperfusion with GG918 increased it by 6.2-fold (0.18%±0.09% vs 1.1%±0.39%).[6] The authors also observed 108-fold higher saquinavir transfer in the fetal-to-maternal direction than from maternal to fetal direction (0.18%±0.09% vs 19.5%±14.5%).

Fatty acids are important nutrients for fetal growth and development. The essential fatty acids for humans are linoleic acid (LA) and a-linoleic acid (ALA). Furthermore, the long-chain polyunsaturated fatty acids (LC-PUFAs) are critically important for fetal brain and retina development. In late pregnancy, enhanced maternal lipid catabolism results in increased availability of LC-PUFAs to the fetus. These LC-PUFAs, such are arachidonic acid (ARA), eicosopentaenoic acid (EPA), and docosahexaenoic acid (DHA) can be synthesized in adults from LA and ALA, but are more readily available from foods of animal origin, particularly seafood. ARA is a precursor for leukotrienes, eicosanoids, and prostaglandins, which also serve as important signaling molecules.[7, 8]

While all of the mechanisms of transplacental fatty acid transport are not completely known, protein-mediated transport is more important physiologically than simple diffusion.[8] In fact, the LC-PUFAs are preferably transported to the fetus, in the order DHA>ARA>AA>OA, in which the saturated fatty acid oleic acid (OA) serves as a comparator.[8] To supply the developing fetus with these important fatty acids, the human placenta contains several fatty acid transport systems. The plasma membranes of placental trophoblast cells express two known isoforms of the Fatty Acid Transporter Protein (FATP), which are FATP1 and FATP4.[8] Meanwhile, the placental syncytiotrophoblast (forming the placental barrier) expresses fatty acid transport proteins 1 and 4 (FATP1 and FATP4, respectively).[9, 10] Of these two transporters, FATP4 is most highly expressed. Therefore, drug delivery by FATP4 may be a viable strategy. However, this would require altering the structure of a drug, by adding a hydrocarbon chain and a free carboxylic acid to make the drug a substrate for transporters such as FATP4.

The drug lopinavir is highly effective against the HIV virus in vitro and is a drug of choice for combating HIV infection. However, lopinavir suffers from poor solubility, poor permeability, high first-pass clearance after oral administration, and low bioavailabity (the actual magnitude of which is unknown). Lopinavir is typically about 98-99% protein bound in vivo. These factors contribute to interpatient variability when lopinavir is used. In particular, poor distribution of lopinavir to HIV sanctuary compartments has been noted. For example, lopinavir fails to cross the placenta and the blood-brain barrier, so that the central nervous system (CNS) and the developing fetus in effect become viral sanctuaries. Strategies to overcome these deficiencies would result in increases in the efficiency of delivery of the drug to HIV sanctuary compartments, thereby reducing latent proviral loads in AIDS patient, and enable additional preinatal treatment options.

A series of novel dicarboxylate esters of lopinavir have been synthesized (e.g. see Example 1). These compounds contain a carboxylic acid moiety, an alkyl chain of varying composition, with another carboxylic acid esterified with the secondary alcohol of lopinavir. The result is a series of compounds which are anionically charged at physiological pH on one end, connected to a bulky, lipophilic group (composed of lopinavir and the alkyl chain). Such compounds display surprising and unexpected uptake characteristics, beyond what would be expected based upon physico-chemical characteristic, such as partition coefficients, hydrogen bonding, and simple diffusional permeability. In particular, such a modification would be expected to decrease diffusional permeability; however, a higher than expected 6-fold increased uptake was observed. Without being bound by theory, the enhanced uptake of the novel compounds may involve the FATP4 fatty acid transporter, which is a transporter heretofore unutilized in drug delivery. This transporter would then facilitate the uptake of the novel compounds from the blood into target tissues, helping drugs like lopinavir reach more effective concentrations in pharmacologic sanctuaries such as the fetal compartment.

In addition, the stability of a prodrug moiety is a critical factor determining the success or failure of any prodrug strategy. Typically, the intact prodrug itself is expected to have little or no pharmacologic activity, as is expected for lopinavir prodrugs. Therefore, the appropriate release of the active compound is critical to obtain therapeutic effects. If the prodrug fails to be hydrolyzed (releasing the active compound), the strategy results in the delivery of an inactive compound. However, if the prodrug is hydrolyzed too readily (ie, prior to absorption) then its delivery will not be improved above the parent compound. As a result, the hydrolysis of the prodrug compounds (spontaneous and/or enzyme-mediated) was also an important consideration and was investigated.[11].

### Synthesis and Testing of Succinyl-³H-Lopinavir (3H-SLPV)

Synthesis and Identification: Using the acid chloride method described below, we obtained 54% yield of succinyl-³H-lopinavir (3H-SLPV) with 98% radiochemical purity. We have also successfully synthesized the unlabelled succinyl-lopinavir, using either this method or other standard esterification methods,[12] by treating lopinavir with N,N-dimethyl-4-aminopyridine (DMAP) and adding either succinic anhydride or succinic acid with dicyclohexylcarbodiimide (DCC). Treatment of this compound with porcine liver esterase followed by HPLC analysis (described below) showed a loss of 3H-SLPV and a corresponding increase in 3H-lopinavir. Recovery of the parent compound shows that SLPV was indeed synthesized, since lopinavir has only one hydroxyl group available for esterification. Additionally, SLPV was dissolved in 50% methanol 50% aqueous ammonium acetate (50 mM pH 5.5) and injected at 20 μL/min into a Micromass ZMD mass spectrometer with atmospheric pressure chemical ionization in positive ion mode. The results indicated the expected molecular ion (M+H) with a mass (m/z) of 729.95 for SLPV, with observations of the expected dehydration product (711.82) and the sodium adduct (751.83). Additionally, SLPV was fragmented to reveal the intact molecular ion (M+H) for lopinavir (629.79) and its dehydration product (611.78). Together, these data demonstrate the feasibility of synthesis and establish the structure of SLPV as the dicarboxylate monoester formed from succinic acid and lopinavir.

HPLC Methods:

A rapid, isocratic HPLC method was developed using a Waters 2695 chromatograph pumping 75% methanol 25% aqueous 50 mM ammonium acetate (pH 5.5) through an Alltech Alltima HP C18 3 μm 4.6×100 mm column at 1 ml/min, and detected using a Waters 2487 detector set at 260 nm. Within a 4.5 min run time, elution times for succinyl-lopinavir (SLPV) and lopinavir (LPV) were 2.5 and 3.9 min (respectively); extraction efficiency was 106+/−16%; calibration curves were linear from 0.2 μM to 20 μM (r2>0.99), thus facilitating analysis over a clinically relevant concentration range. Thus, this method is a useful, novel, sensitive, and rapid analytical method to quantitate both LPV and its dicarboxylate monoesters.

Stability:

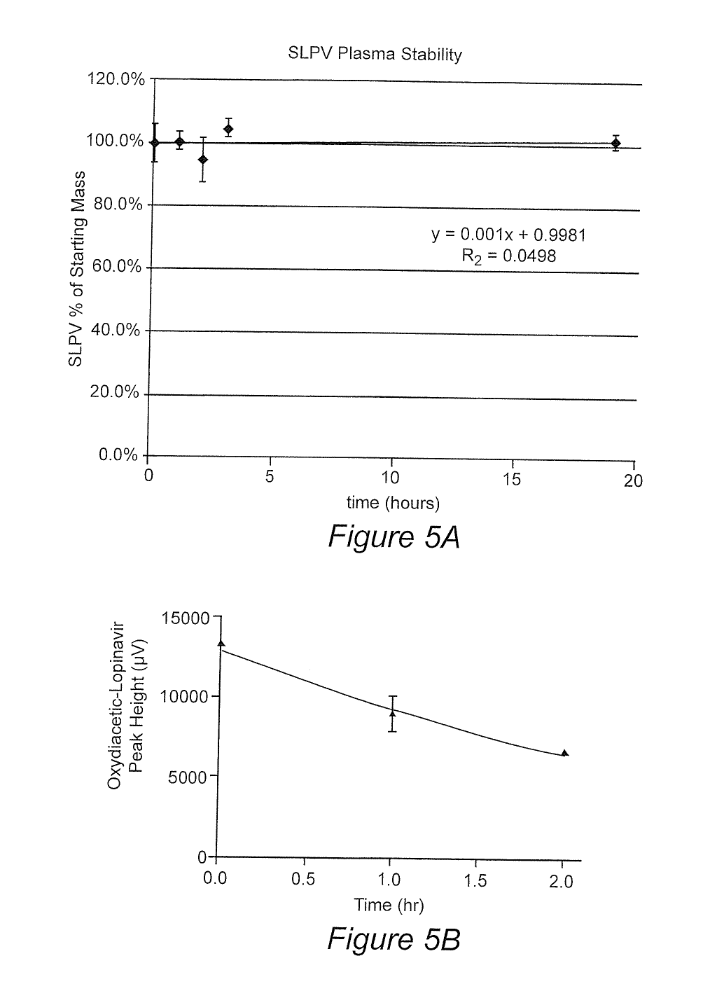

The stability of SLPV was determined by incubating SLPV (20 μM) in human plasma from one healthy adult at 37° C. for 18 hours. The results showed that SLPV is completely stable at physiological pH and is not significantly degraded by plasma esterases (FIG. 5A). However, another dicarboxylic acid monoester of lopinavir has been synthesized, called oxydiacetic-lopinavir. Since free oxydiacetic acid has a lower calculated pKa than free succinic acid (2.73 vs. 4.74), we expected oxydiacetic lopinavir to be more easily hydrolyzed, releasing free lopinavir to exert its antiretroviral activity. Indeed, this was the case, and our preliminary data showed that oxydiacetic lopinavir has a plasma hydrolysis half-life of approximately 2 hours (FIG. 5B). Thus, it is possible to modulate the stability of lopinavir dicarboxylate monoesters by varying certain characteristics of the dicarboxylate,

Uptake of SLPV in BeWo Cells and Primary Human Cytotrophoblast Cells:

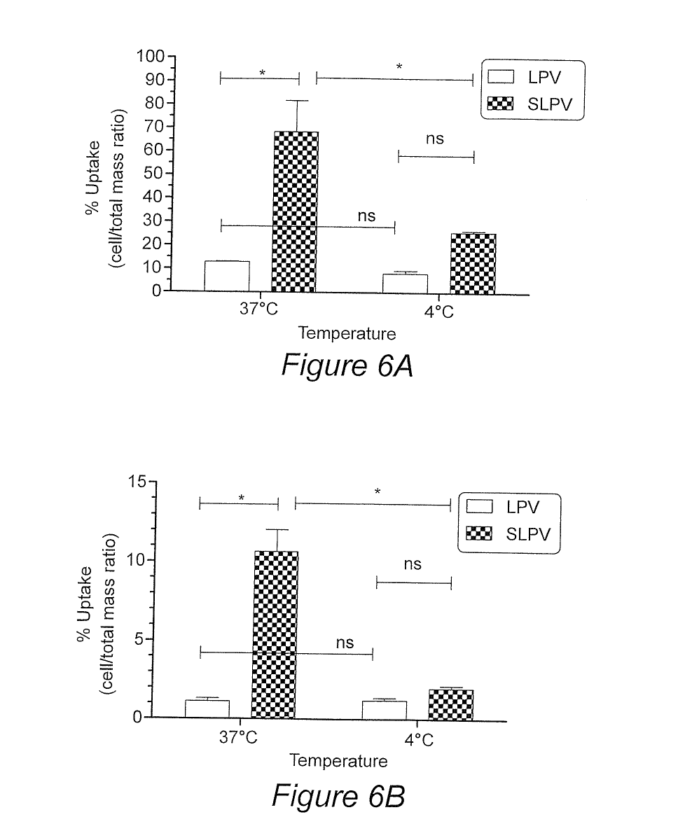

Using the BeWo cell line, we compared the uptake of LPV and 3H-SLPV at 37° C. and 4° C. As shown in FIGS. 6A and B, uptake of SLPV was greatly enhanced compare to Lop. Furthermore, SLPV uptake was inhibited at reduced temperatures, whereas LPV uptake was not significantly reduced.

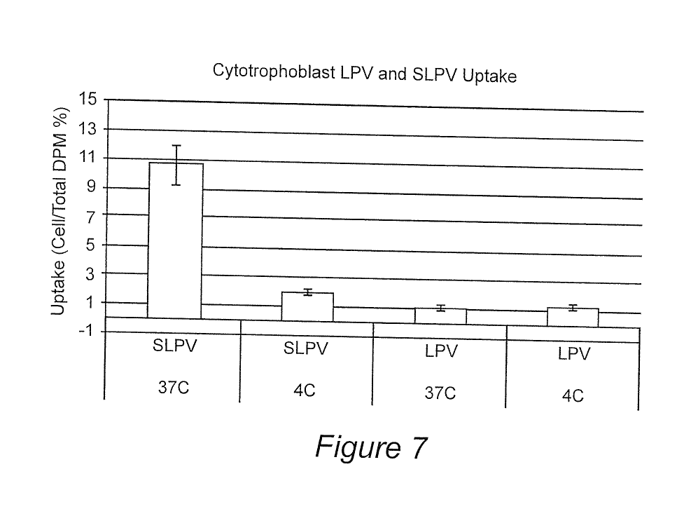

We also isolated primary cytotrophoblast cells from one term (39 weeks gestation) placenta delivered by cesarean section. Cells were cultured in DMEM and uptake of ³H-SLPV and ³H-LPV were determined after 24 hrs in culture as described. The results showed that SLPV uptake by human placental cytotrophoblasts at 37° C. was 10-fold greater than uptake of LPV (FIG. 7). Furthermore, the uptake of SLPV was temperature-dependent as shown by the inhibition of uptake at 4° C. (FIG. 7). These results are consistent with those observed in BeWo cells, and indicate that uptake of SLPV is a transporter-mediated process in contrast to LPV uptake in which diffusion appears to dominate. These results also demonstrate the feasibility of isolating cytotrophoblast cells, and the similarity in behavior of cytotrophoblasts and BeWo cells with respect to SLPV and LPV.

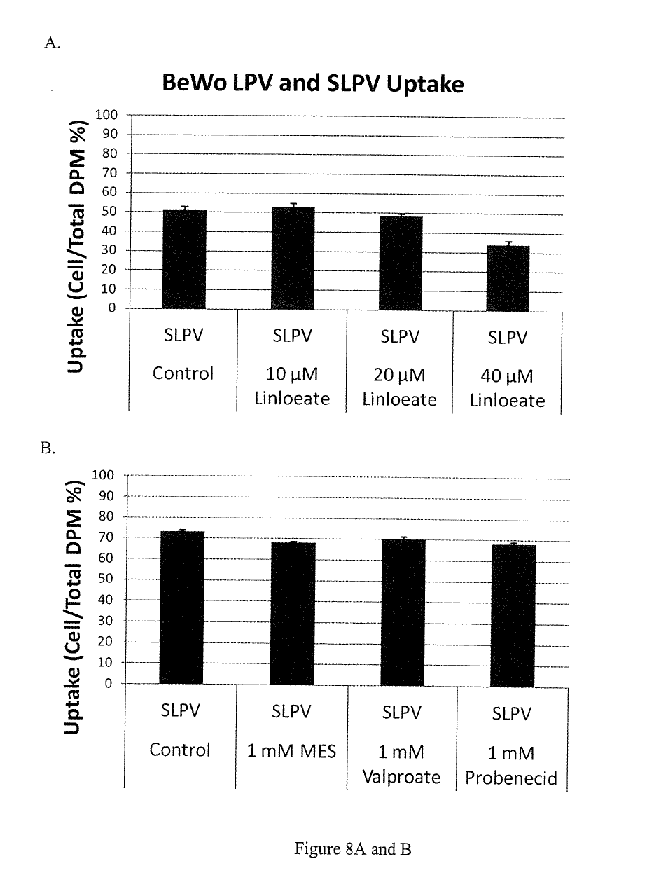

Since SLPV is a large (MWt=728.87) lipophilic molecule with a free carboxylic acid group, SLPV may be a substrate for fatty acid transporters. Linoleic acid has previously been demonstrated to be preferentially transported in the apical-to-basolateral direction (analogous to the maternal-to-fetal direction).[13] We found uptake of S-³H-LPV in the presence of 40 μM linoleic acid (LA) was significantly reduced by 34% in BeWo cells (p<0.05, FIG. 8A), whereas LPV uptake was unaffected (not shown). In addition, uptake was only slightly reduced by 1.0 mM monoethyl succinate and 1.0 mM probenecid, but not by 1.0 mM sodium valproate, all of which are known organic anion-transporting (OAT) inhibitors (MCTs, gene family SLC22A) (FIG. 8B). Interestingly, the ATP-binding cassette (ABC) transporter inhibitor MK571 caused a slight increase in uptake (not shown). In addition, no effect on uptake was observed when SLPV was tested in the presence of 100 μM taurocholate, a classic subtrate for bile salt transporters, including the sodium-dependent taurocholate transporter (NTCP; gene family SLC10A1), the apical bile salt transporter (ASBT, gene family SLC10A2), and certain organic anion-transporting (OATPs, gene family SLC22A); and 40 arachidonic acid and 50 μM docosahexaenoic acid, inhibitors of the uptake of very long chain polyunsaturated fatty acids. The addition of bromosulphophthalein (BSP), an inhibitor or several organic anion transporters (gene families SLC22A and SLCO), also did not influence uptake, even at 250 μM. Representative data is shown in Table 1. Together, these results support the hypothesis that LPV dicarboxylate monoesters are substrates for fatty acid transporters (such as FATP4) which are expressed in the placental trophoblast cells.

TABLE 1
Uptake of lopinavir and succinyl-lopinavir in the presence
of various transport inhibitors.

| | |
|---|---|
| Extent of Uptake Inhibition | Substrate |

| Inhibitor | Lopinavir | Succinyl-lopiniavir |
|---|---|---|
| Cold (4° C.) | 10% | 62% |
| BSP (250 μM) | 7% | 7% |
| taurocholate (100 μM) | 9% | −1% |
| monoethyl succinate (1 mM) | | 7% |
| valproic acid (1 mM) | | 5% |
| probenecid (1 mM) | | 7% |
| linoleic acid (40 mM) | 9% | 40% |

Additional experiments are designed which identify the lopinavir dicarboxylate ester exhibiting the highest uptake/transport in an in vitro model of the human placental epithelium. The results show that dicarboxylate monoesterification of LPV increases its placental uptake/transport, mediated by FATP4. A series of LPV esters are synthesized in which dicarboxylate chain length, unsaturation, pKa, and electronegative functional groups are varied. The compounds are tested to determine their transport activities in the BeWo model. The BeWo human trophoblast cell culture model is commonly used for studying uptake and transcellular permeability across the placental barrier, and is used to determine the transport activities of the LPV esters. The initial data (see above) indicated the greatly enhanced uptake of lead compound SLPV. These additional studies result in the identification of the LPV ester with highest uptake transport in these in vitro barrier models, and this compound is chosen for further testing.

In addition, experiments are designed to establish the increased placental delivery of the lopinavir dicarboxylate ester over lopinavir using ex vivo tissue models, to demonstrate that the LPV dicarboxylate monoester enables greater penetration across membrane barriers expressing FATP4, compared to LPV. The focus is on the placental barrier as the hurdle generating this pharmacologic sanctuary. This tissue has a tight-junction cell layer and expresses the ABC efflux transporters, thereby restricting the entrance of many compounds, especially HIV protease inhibitors. However, it also expresses FATP4, thus providing a window of opportunity to deliver drugs to this tissue by “disguising” them as FATP4 substrates. The directional (maternal to fetal vs. fetal to maternal) transport of LPV and the ester is determined using the isolated perfused human placental cotyledon model. These studies establish the in vivo feasibility of the lopinavir dicarboxylate ester approach to deliver lopinavir into the fetal compartments.

### Identification of the Lopinavir Dicarboxylate Ester Exhibiting the Highest Uptake/Transport in an In Vitro Model of the Placental Epithelium.

### Chemical Synthesis

LPV esters were or are synthesized from lopinavir powder (BetaPharma, Inc) by dissolving lopinavir (100 μmol) and N,N-dimethyl-4-aminopyridine (DMAP; 130 μmol) in anhydrous dimethylformamide (DMF). Separately, the dicarboxylic acids (200 μmol) and EDC.HCl (220 μmol) were or are mixed in anhydrous DMF, under inert atmosphere over 4 Å molecular sieves in a total reaction volume of 1 ml. The two resulting solutions were or are stirred under inert atmosphere at room temperature for 48-72 hours, and reaction progress was or is monitored by reversed-phase HPLC with gradient elution and detection by UV (254 nm) and fluorescence (ex 266 nm, em 300 nm). Upon completion, the reaction was or is evaporated under reduced pressure, reconstituted with methanol/aqueous acetic acid, and purified by preparative-scale reversed-phase (C-18, 4 g) flash chromatography (Agela, Inc) with reduced-pressure evaporation of eluted fractions to achieve>95% chemical purity. ³H-LPV dicarboxylate esters were or are synthesized from ³H-lopinavir (Moravek, Inc.) by drying 100 μCi (typically ˜1 mop and reacting with dicarboxylic acids (Table 1) in the presence of excess solvent. The reaction product was or is worked up as above, but with purification on an Alltima HP C18 4.6×100 mm 3 μm column, with evaporation of eluted fractions to achieve radiochemical purity>97%. With reference to Table 2, lopinavir esters of carboxylates 1, 2, 4, 5 (also referred to as thioglycolic acid), 6, 8 (also referred to as diglycolic acid) have already been synthesized, as have esters of adipic acid, 2-ketoglutaric acid and 3-ketoglutaric acid. In addition, esters of 8 and ritonavir, saquinavir, nelfnavir, indinavir, atazanavir and tipreanvir have been synthesized and purified. Lopinavir esters 1 and 8 have been purified, and lopinavir esters of 1 have been tested as described herein. Table 3 provides a list of compounds which have been synthesized and the status of testing.

TABLE 2
Carboxylic acids for attachment to lopinavir, or other similar drugs.

| | | | | | Other |
|---|---|---|---|---|---|
| | | | | | electro- |
| | | Calculated | Chain | # of Un- | negative |
| # | Acids | pKa | length | saturations | groups |
| 1 | succinic | 4.24 | C4 | | |
| 2 | thiodipropionic | 4.03 | C3-S-C3 | | thioether |
| 3 | dihydromuconic | 3.96 | C6 | 1 | |
| 4 | muconic | 3.77 | C6 | 2 | |
| 5 | thiodiacetic acid | 3.25 | C2-S-C2 | | thioether |
| 6 | fumaric | 3.15 | C4 | 1 | |
| 7 | malonic | 2.92 | C3 | | |
| 8 | oxydiacetic | 2.73 | C2-O-C2 | | ether |
| 9 | maleic | 2.39 | C4 | 1 | |
| 10 | furandicarboxylic | 2.28 | C6 | 2 (aromatic) | furan |
| 11 | glutaric | 4.33 | C5 | | |
| 12 | adipic | 4.39 | C6 | | |
| 13 | suberic | 4.46 | C8 | | |

TABLE 3
Status of synthesis and testing of representative prodrugs

| Dicarboxylic | HIV PROTEASE INHIBITORS | | | | | | |
|---|---|---|---|---|---|---|---|
| acids | lopinavir | ritonavir | saquinavir | nelfinavir | indinavir | atazanavir | tipranavir |
| succinic | ++ | | | | | | |
| diglycolic | ++ | +* | +* | + | + | + | + |
| thiodiglycolic | + | | | | | | |
| glutaric | ++ | | | | | | |
| maleic | + | | | | | | |
| fumaric | + | | | | | | |
| adipic | ++ | | | | | | |
| 2-ketoglutaric | + | | | | | | |
| 3-ketoglutaric | + | | | | | | |
| thiodipropionic | + | | | | | | |
| muconic | + | | | | | | |

+ indicates compounds which have been synthesized;
++ indicates a compound with biological activity;
+* indicates partial characterization of biological activity.

### Chemical Identification:

To determine the chemical identity of the esters compounds were or are dissolved in 80% acetonitrile/20% aqueous (0.1% trifluoroacetic acid) and infused onto a Waters Micromass ZMD mass spectrometer, with atmospheric pressure chemical ionization in positive ion mode, and monitoring from 300 to 800 m/z. Spectra of products were or are compared to solvent alone or lopinavir to determine the molecular ion. Finally, to confirm the structure of lopinavir esters, compounds were or are reconstituted with CDCl₃ for NMR analysis. The identity of lopinavir esters was or is confirmed by proton NMR spectrometry using, e.g. a Varian Mercury 300-MHz spectrometer. ³H-LPV esters were or are identified by HPLC retention time using the unlabelled ester as an authentic standard.

### Cell Culture Studies:

BeWo cells are a human trophoblast cell culture model of the human syncytiotrophoblast, which can be easily cultured, and can form tight junctions when plated on Transwell filters, thus permitting directional transport experiments. Initial data showed that BeWo cells behave similarly to primary human placental cytotrophoblasts in terms of the degree of enhanced SLPV (vs. LPV) transport. Therefore, this model is used to determine which dicarboxylate monoester of LPV has the greatest uptake activity.

The BeWo cell line is originally derived from a human choriocarcinoma. The BeWo cell line (Schwartz clone; passage 30) was a gift. BeWo cells in these studies is used between passages 30-70. BeWo cells are cultured in high glucose Dulbecco's modified Eagle's medium (DMEM) supplemented with 10% heat inactivated fetal bovine serum (FBS).

### Determination of Transmonolayer Permeability:

BeWo cells used in this study cells are seeded onto 12-well transwell inserts (0.4 μM pore size) at a density of 80,000 cells/cm². Transepithelial electrical resistance (TEER) values are determined and corrected for resistance of the collagen coated filters in the absence of cells. The cells are loaded with the compounds (10 μM) in the apical chamber for apical-to-basolateral studies, and in the basolateral chamber for basolateral-to-apical studies. The receiver chamber contains transport buffer (HBSS+HEPES, pH 7.4) without drugs added. Transwells are incubated at 37° C. with shaking (50 r.p.m). Aliquots (200 μL) are removed from the receiver chambers at pre-determined time points (up to 2 hours), and replaced with an equal volume of pre-warmed transport medium. 25 μL acetonitrile are added to the withdrawn samples, centrifuged, and a portion of the supernatant is used for analysis by HPLC as described below.

### Increased Placental Delivery of the Lopinavir Dicarboxylate Ester Over Lopinavir Using Ex Vivo Tissue Models.

Isolated Perfused Placental Cotyledon:

The isolated perfused placental cotyledon model is used to establish SLPV transport in the intact functional unit of the placenta, known as the cotyledon. In each case, 3-5 separate experiments are performed. To compare the transplacental transport of LPV and SLPV, the dually perfused isolated placental cotyledon model is used.[14] In this technique, 3H-LPV or 3H-SLPV (10 nCi/ml) is added to either the maternal or fetal perfusion fluid, and the passage of the compounds to the opposite side is examined. Antipyrine is added as a passive permeability marker and assayed by HPLC as described.[15] Fluid shift is measured to determine leakiness. Buffers are gased, filtered, re-warmed, and recirculated. Samples are taken every 10 minutes for 2 hours, and the samples are evaporated, reconstituted with 50/50 methanol/aqueous acetic acid (1%), and analyzed by HPLC with radiomatic detection as described below. Mass transfer is plotted vs time, and the clearance index relative to antipyrine are determined, as previously described.[16] 5 perfusions for each compound are performed (total=10 placentae).

### Hydrolysis and Binding of LPV Esters in Plasma and Placental Tissue:

In order to release the active (free) lopinavir, the ester bond must be hydrolyzed. Therefore, the relationship between the pKa of dicarboxylic acids and their rates of hydrolysis is investigated, in order to achieve a more effective delivery of free lopinavir. The ideal prodrug compound should be stable enough to permit the target tissue (i.e., placenta) to take up the prodrug, achieve reasonable initial fetal blood concentrations (approaching or exceeding that of lopinavir), and releasing the active lopinavir, so that it is possible to increase the fetal lopinavir exposure. To examine the hydrolysis of lopinavir esters, human plasma and human placental villous tissue homogenate are incubated with lopinavir ester prodrugs at clinically relevant concentrations (0.1-10 μM) with sampling at various time points (0-8 hours) Appropriate esters such as acetylsalicylic acid and 4-methylumbelliferyl acetate are used as positive controls for esterase activity. The effects of esterase inhibitors (e.g., BNPP; 100 μM) are determined. Furthermore, binding of lopinavir prodrugs to proteins in plasma or placental homogenate is determined in the presence or absence of BNPP. Lopinavir dicarboxylate esters have a lower degree of protein binding compared to lopinavir, due to their negative charge and their higher degree of aqueous solubility.

### Analysis of LPV and LPV Esters:

To determine the concentrations of LPV and LPV esters, protein is precipitated by adding two volumes of cold methanol/acetic acid (99:1) followed by centrifugation. The samples are then separated by reversed phase HPLC, eluted isocratically with 65% methanol/35% aqueous (1%) acetic acid, and detected by UV (λ=260 nm) and fluorescence (λex=266 nm; λem=300 nm). To quantitate 3H-SLPV and 3H-Lop, samples will be treated as above, and separated by HPLC as above with liquid scintillation counting of the eluted fractions. This method gives excellent specificity and sensitivity to concentrations between 0.03-100 μM, thus facilitating the proposed studies. The method also allows the separate determination of the ester prodrugs and the parent compound (lopinavir). Mass spectrometry is used to analyze non-radiolabelled lopinavir and ester prodrugs as an alternate method of analysis.

### Statistical Analysis:

Data from inhibitor studies is compared using one-way ANOVA with Dunnett's post test. Data comparing various parameters determined for SLPV and LPV is determined by two-tailed unpaired t-test, except where mentioned.

### RESULTS

The results of this study are as follows. The dicarboxylate monoester of lopinavir with best transport properties in the cell culture models of the human placental barrier is established. The stability of the compounds in human plasma is investigated. Enhanced transport of the optimal compound across the isolated perfused placental cotyledon is demonstrated. These studies result in the identification of the LPV ester with highest uptake transport in these in vitro barrier models, and confirm the lopinavir dicarboxylate ester approach to deliver lopinavir across the placenta, into the fetal compartment.

The results of this study provide “proof of concept” that fatty acid transporters such as FATP4 can be utilized as a novel drug delivery mechanism into protected compartments (pharmacologic sanctuaries) such as the fetal compartment. The methods are useful in achieving higher fetal blood concentrations of HIV protease inhibitors, providing better protection against HIV vertical transmission. Such a strategy shifts the paradigm for treatment of diseases for which it is difficult to transport a drug across other barriers such as the blood-brain barrier, which also highly expresses FATP4. For example, this approach eliminates the brain as an HIV viral sanctuary, and prevents (or treats) HIV viral encephalitis in AIDS patients, and is useful for brain cancer treatment as well.

### REFERENCES FOR EXAMPLE 3

1. Libman H, Makadon H J: Transmission, Pathogenesis, and Natural History. HIV, 3rd ed: American College of Physicians, 2007; 1-34.
2. Gulati A, Gerk P M: Role of placental ATP-binding cassette (ABC) transporters in antiretroviral therapy during pregnancy. J Pharm Sci 2009; 98(7): 2317-35.
3. Watts D H: Treating HIV during pregnancy: an update on safety issues. Drug Saf 2006; 29(6): 467-90.
4. Lankas G R, Wise L D, Cartwright M E, Pippert T, Umbenhauer D R: Placental P-glycoprotein deficiency enhances susceptibility to chemically induced birth defects in mice. Reprod Toxicol 1998; 12(4): 457-63.
5. Smit J W, Huisman M T, van Tellingen O, Wiltshire H R, Schinkel A H: Absence or pharmacological blocking of placental P-glycoprotein profoundly increases fetal drug exposure. J Clin Invest 1999; 104(10): 1441-7.
6. Molsa M, Heikkinen T, Hakkola J, et al.: Functional role of P-glycoprotein in the human blood-placental barrier. Clin Pharmacol Ther 2005; 78(2): 123-31.
7. Beare-Rogers J, Dieffenbacher A, Holm J V: Lexicon of Lipid Nutrition (IUPAC Technical Report). Pure Appl Chem 2001; 73(4): 685-744.
8. Duttaroy A K: Transport of fatty acids across the human placenta: a review. Prog Lipid Res 2009; 48(1): 52-61.
9. Qi K, Hall M, Deckelbaum R J: Long-chain polyunsaturated fatty acid accretion in brain. Curr Opin Clin Nutr Metab Care 2002; 5(2): 133-8.
10. Larque E, Krauss-Etschmann S, Campoy C, et al.: Docosahexaenoic acid supply in pregnancy affects placental expression of fatty acid transport proteins. Am J Clin Nutr 2006; 84(4): 853-861.
11. Beaumont K, Webster R, Gardner I, Dack K: Design of ester prodrugs to enhance oral absorption of poorly permeable compounds: challenges to the discovery scientist. Curr Drug Metab 2003; 4(6): 461-85.
12. Neises B, Steglich W: Simple Method for the Esterification of Carboxylic Acids. Angewandte Chemie International Edition in English 1978; 17(7): 522-524.
13. Liu F, Soares M J, Audus K L: Permeability properties of monolayers of the human trophoblast cell line BeWo. Am J Physiol 1997; 273(5 Pt 1): C1596-604.
14. Walsh S W, Vaughan J E, Wang Y, Roberts L J, 2nd: Placental isoprostane is significantly increased in preeclampsia. FASEB J 2000; 14(10): 1289-96.
15. Zuo M, Duan G L, Ge Z G: Simultaneous determination of ropivacaine and antipyrine by high performance liquid chromatography and its application to the in vitro transplacental study. Biomed Chromatogr 2004; 18(9): 752-5.
16. Sudhakaran S, Ghabrial H, Nation R L, et al.: Differential bidirectional transfer of indinavir in the isolated perfused human placenta. Antimicrob Agents Chemother 2005; 49(3): 1023-8.

### Example 4

### Treatment of HIV Infection: Prodrug Delivery to the Brain

HIV infection needs no introduction as a serious world health problem, which continues despite many advances in its prevention and treatment. The existence of HIV viral sanctuaries in the central nervous system (CNS; e.g., brain) and the gut-associated lymphatic tissue (GALT) is one of the problems which helps the virus to establish the initial infection and to evade eradication. In a word, HIV “hides” in these viral sanctuaries, escaping the immune response and avoiding antiviral medications. The consequence is that the virus successfully infects the patient, and can eventually develop resistance to the medications and cause HIV viral encephalopathy which destroys the brain. The present invention provides antiretroviral drugs which are targeted to reach the HIV viral sanctuaries and destroy the ability of the virus to hide, by designing antiretroviral prodrugs which follow the body's pathways for fatty acid nutrients.

To treat HIV infected patients, multiple antiretroviral drugs are used in what is known as Highly Active Antiretroviral Therapy (HAART). However, the drugs used in HAART therapy (such as protease inhibitors like lopinavir) fail to reach CNS and GALT in adequate concentrations, thus accounting for their inefficacy in eradicating HIV from these viral sanctuaries. To overcome this inefficiency, the present invention utilizes endogenous fatty acid transporters (FATP) as a drug delivery mechanism. The physiologic role of various fatty acid transport processes in the uptake and disposition of nutrients into the CNS and GALT is well established in the literature. However, the FATPs have not previously been considered as a drug delivery route. In addition, the use of these pathways enables a drug to simultaneously reach both the CNS and the GALT. The HIV viral load is very high in the CNS and GALT of newly-infected HIV patients or patients receiving the standard highly active antiretroviral therapy (HAART). Therefore, the strategy described herein is to alter drugs like lopinavir so that they can use the FATP transporters to enter the CNS and GALT as nutrients do. Fatty acid transporters are expressed in the CNS and GALT, and the compounds of the invention are substrates for fatty acid transporters and are thus taken us by FATP.

Many previous attempts to increase delivery of drugs to the brain have been only moderately (or not at all) successful. The brain capillary endothelium (forming the blood-brain barrier) is known to express fatty acid transport proteins 1 and 4 (FATP1 and FATP4, respectively). Of these two transporters, FATP4 is the most highly expressed. Therefore, drug delivery by FATP4 is a viable strategy once the structure of the drug is altered by adding a hydrocarbon chain and a free carboxylic acid to make the drug a substrate for transporters such as FATP4.

Accordingly, a series of novel dicarboxylate esters of lopinavir have been or are synthesized. These compounds, contain a carboxylic acid moiety, an alkyl chain of varying composition, with another carboxylic acid esterified with the secondary alcohol of lopinavir.

The result is a series of compounds which are anionically charged at physiological pH on one end, connected to a bulky, lipophilic group (composed of lopinavir and the alkyl chain). Such compounds display surprising and unexpected uptake characteristics, beyond what would be expected based upon physico-chemical characteristics, such as partition coefficients, hydrogen bonding, and simple diffusional permeability. While such a modification may be expected to decrease diffusional permeability somewhat, we found a 6-fold increased uptake, far greated than can be accounted for by diffusion. With out being bound by theory, the enhanced uptake of the synthesized compounds may involve the FATP4 fatty acid transporter, which is a transporter heretofore unutilized in drug delivery. This transporter likely facilitates the uptake of the prodrug compounds from the blood into target tissues, helping drugs like lopinavir reach more effective concentrations in pharmacologic sanctuaries such as the brain and fetal compartments. This strategy can be applied to many anti-retroviral compounds.

### Design and Synthesize the Lopinavir Dicarboxylate Esters as Novel Fatty Acid Transporter Substrates.

Carboxylate monoesters of lopinavir (LPV) are designed and tested for best transport properties in cell culture models of the human blood-brain barrier. This establishes which carboxylate monoesters of lopinavir increase its brain uptake/transport, mediated by FATP4. A series of lopinavir esters is synthesized by varying carboxylate chain length, level of unsaturation, pKa, and electronegativity of functional groups. LPV esters are synthesized using standard esterification reactions. The structures of the compounds are determined and/or confirmed by mass spectrometry and nuclear magnetic resonance.

In order to release the active (free) lopinavir, the ester bond must be hydrolyzed. Therefore, the relationship between the pKa of dicarboxylic acids and their rates of hydrolysis when present in the prodrugs are determined, in order to achieve effective delivery of free lopinavir. The stability of the compounds in human plasma is determined by measuring both the disappearance of the ester and the appearance of lopinavir as a function of time and temperature. The preferred compounds are stable in plasma for a time between 20 to 120 minutes.

Increased delivery of the lopinavir dicarboxylate ester to HIV viral sanctuaries is established using in vitro models using the blood-brain barrier as a model of the CNS HIV viral sanctuary. The blood-brain barrier has tight-junction cell layers and expresses the ABC efflux transporters, thereby restricting the entrance of many compounds, especially HIV protease inhibitors. However, it also expresses FATP4, thus providing a window for delivery of drugs to these tissues by “disguising” them as FATP4 substrates.

The prodrug compounds are tested to determine their transport activities in vitro using the CMEC/D3 human brain capillary endothelial cell culture model as a new and unique tool for studies of the human blood-brain barrier penetration. This model is used to determine uptake and transcellular permeability for the lopinavir esters. The cells are grown on filters, where they form a barrier between two fluid compartments representing the brain and the blood. The compounds are added to one fluid compartment, and their appearance on the other compartment is determined. While lopinavir crosses very slowly; the prodrugs of the invention (as FATP4 substrates) cross quickly. This model is used to determine the directional (blood to brain vs. brain to blood) transport of lopinavir and the prodrug ester and establish the feasibility of the lopinavir dicarboxylate ester approach to deliver lopinavir into the brain.

Initial data indicated greatly enhanced uptake of lead compound SLPV (see Example 2). The studies described in this Example identify the lopinavir ester with highest uptake and transport in in vitro blood-brain barrier models, and this compound is selected for in vivo testing.

These studies establish lopinavir dicarboxylate monoesters as substrates of FATP transporters at the blood-brain barrier. The studies also establish the release of free (active) lopinavir in the tissues. Finally, the studies provide an optimized compound for testing in animal models Such a strategy shifts the paradigm for treatment of diseases for which it is difficult to deliver anti-HIV drugs into these tissues. For example, this approach eliminates the brain as an HIV viral sanctuary, and prevents (or treats) HIV viral encephalitis in AIDS patients. It is also be useful in achieving higher fetal blood concentrations of HIV protease inhibitors, providing better protection against HIV vertical transmission.

Finally, the efficacy of the prodrugs is tested in animal models. First, the rat model is used to characterize the pharmacokinetic disposition of the prodrugs, demonstrating their delivery into the CNS and GALT tissues and their release of active lopinavir. The efficacy of the prodrugs are also tested in the SIV-infected macaque model.

Clinical studies in humans are carried out upon proper registration with appropriate government agencies.

### Example 5

### Investigations of Diglycolic-Lopinavir (DGLPV)

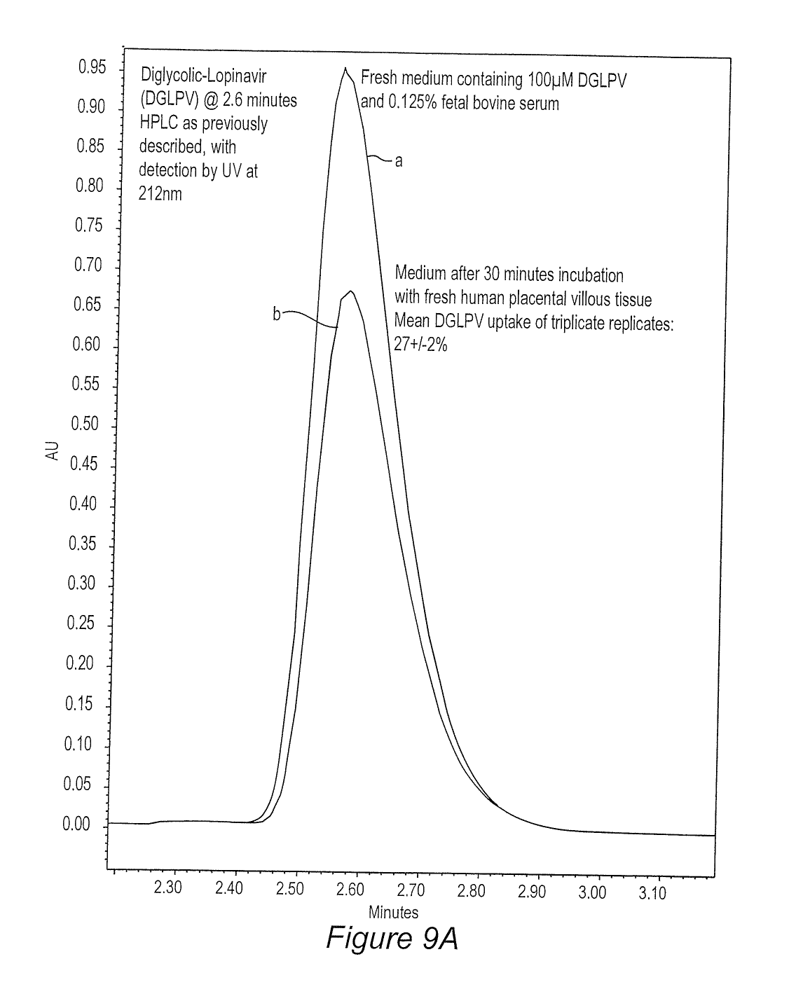

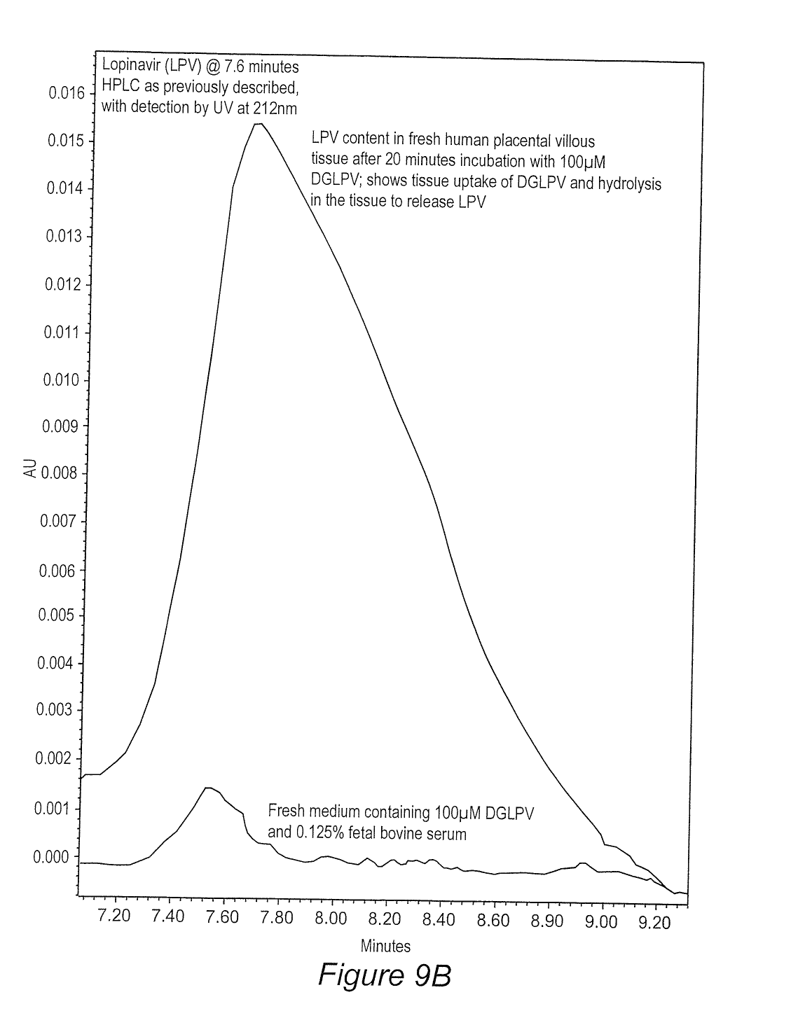

DGLPV was synthesized as described above. The amount of DGLPV in the media originally containing 100 uM DGLPV was measured using HPLC as described above, and again after a 30 minute incubation with fresh human placental villous tissue in the same media. The results are presented in FIG. 9A, which shows a decrease in DGLPV in the medium.

To confirm that the DGLPV was taken up by the tissue and hydrolyzed therein, HPLC was used to measure the amount of free LPV present in the tissue. The results are presented in FIG. 9B. As can be seen, the control (fresh media containing 100 uM DGLPV) had almost no free LPV present. In contrast, the results showed an approximately 15-fold enrichment of free LPV in the placental tissue which has been incubated with DGLPV.

This example shows that like SLPV, DGLPV can also be transported into human tissues. However, unlike SLPV, DGLPV can also be hydrolyzed in the tissue to release the free (active) lopinavir.

### Example 6

### Further Investigations on LPV Esters

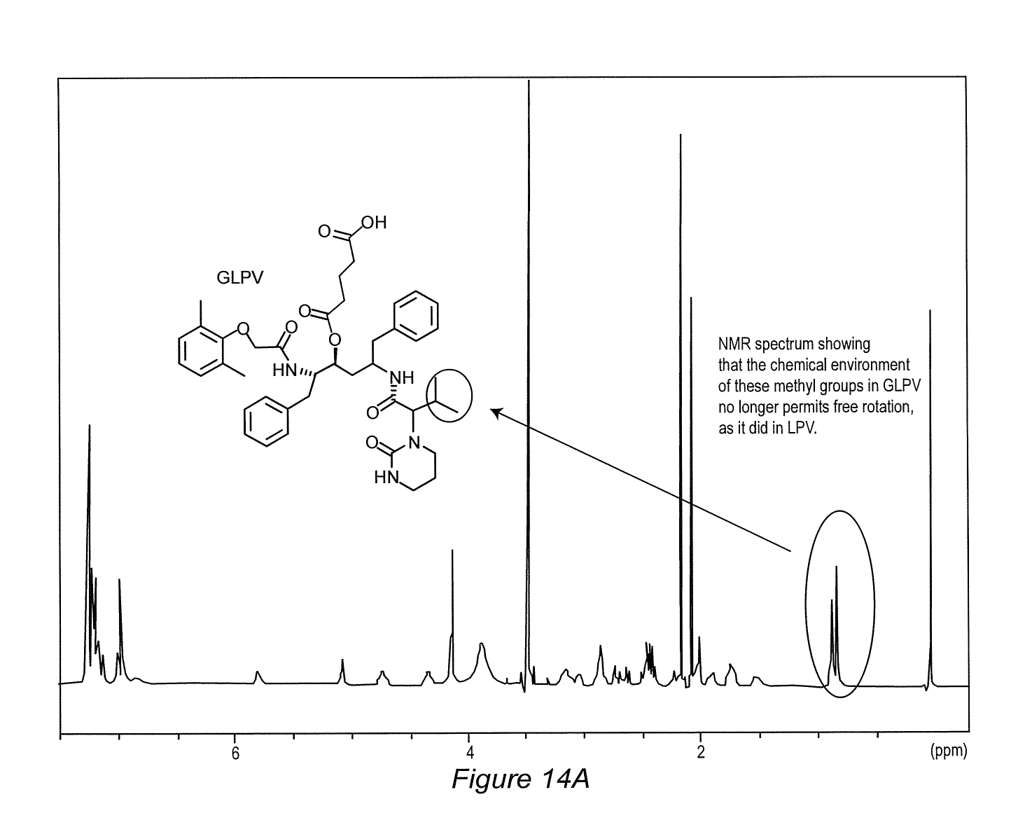

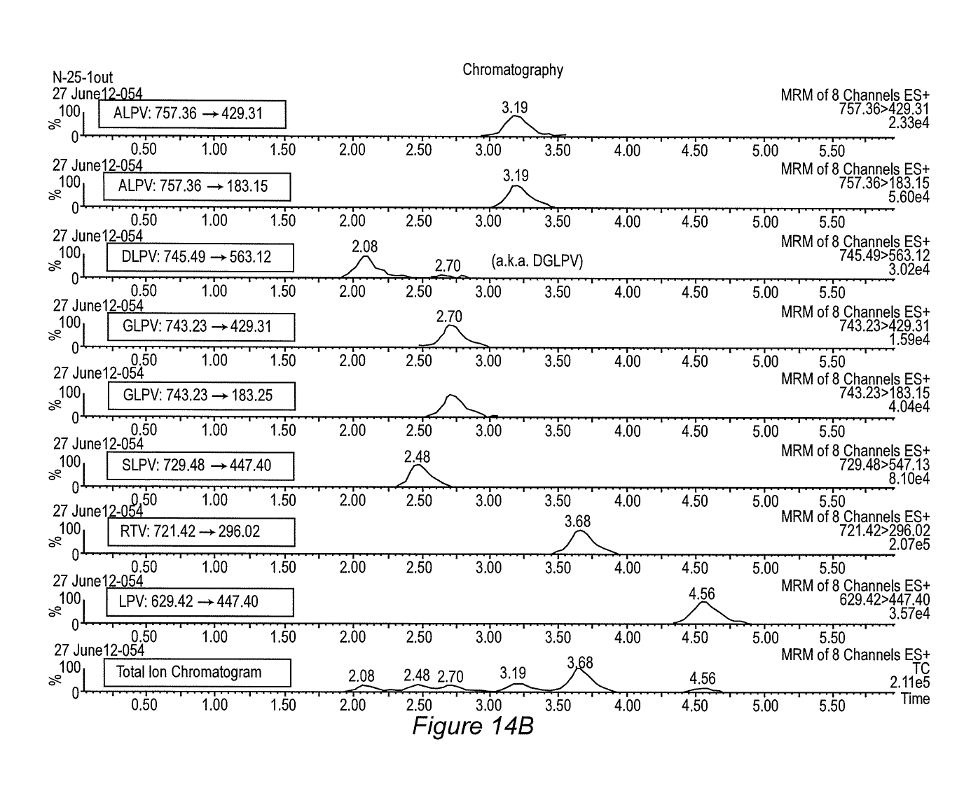

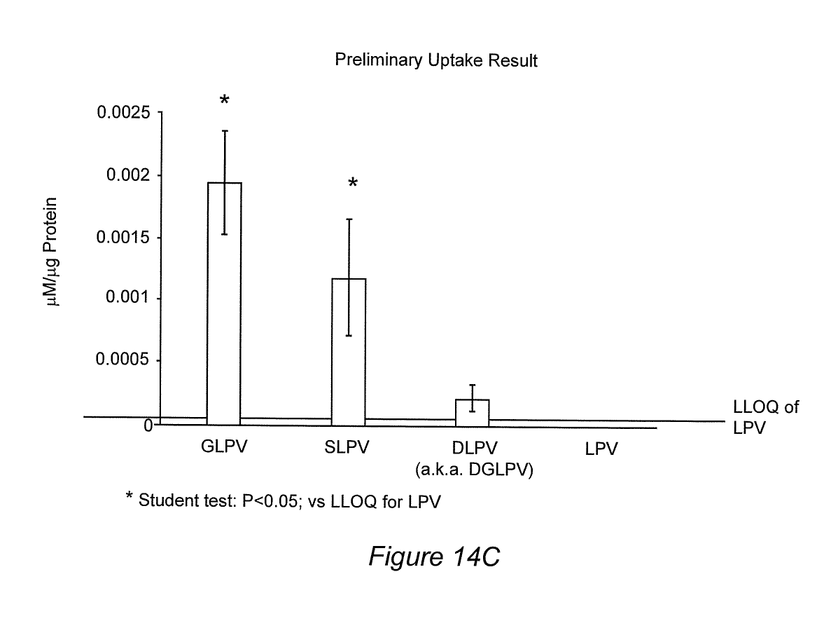

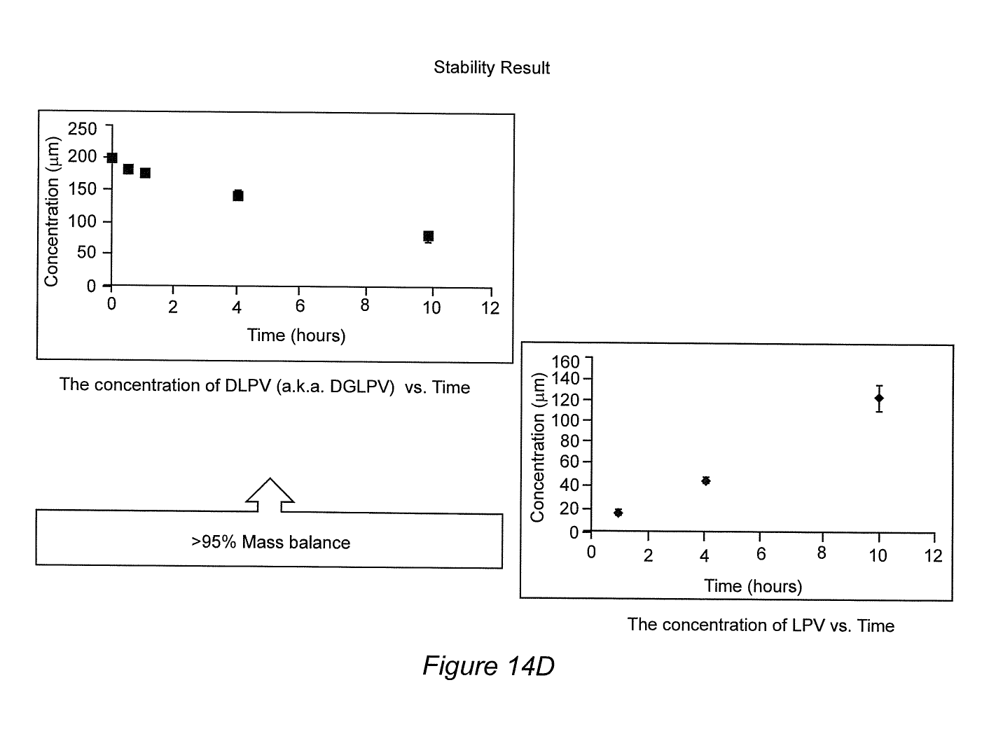

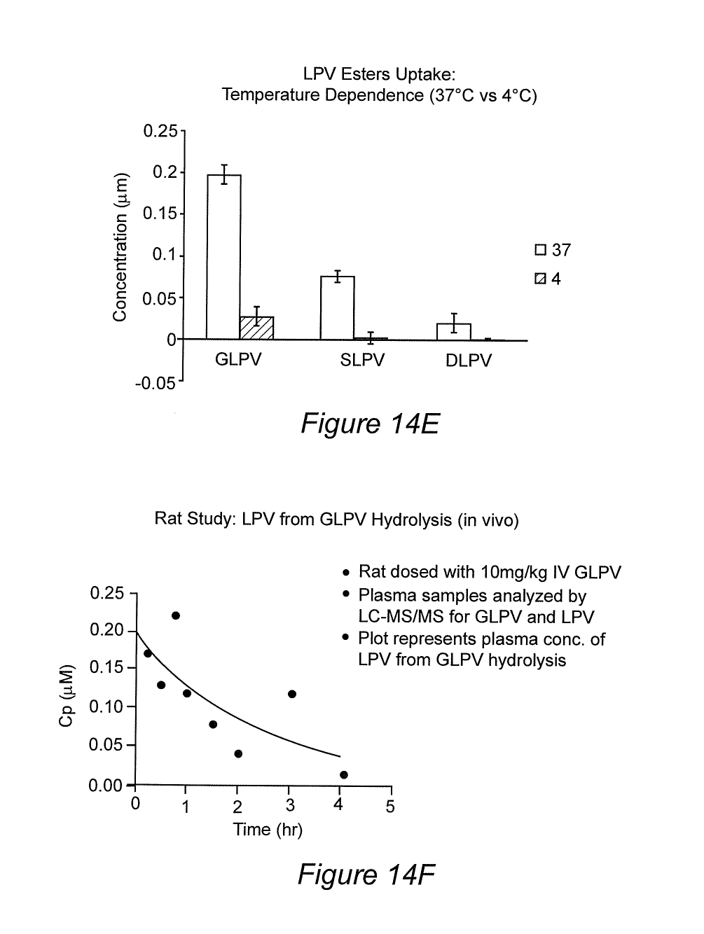

NMR spectroscopy was performed on the compounds including GLPV (FIG. 14A). The NMR spectrum shows that the chemical environment of an isopropyl group of GLPV no longer permits free rotation, as was seen in LPV spectrum. An LC-MS/MS assay was developed and validated to determine concentrations of the novel compounds in biological matrices and fluids, as shown in FIG. 14B. This assay was used to determine the uptake of non-radiolabelled LPV esters (GLPV, SLPV, and DLPV) in BeWo cells (FIGS. 14C and E), their stability in plasma (FIG. 14D), and their hydrolysis in vivo in rats (FIG. 14F). The results show that uptake of GLPV>SLPV>DGLPV. The figures also show that uptake of GLPV, SLPV, and DGLPV are all temperature dependent, consistent with a fatty acid transporter-mediated uptake mechanism. Finally, the results show that DGLPV and GLPV are capable of being hydrolyzed in vivo.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A prodrug, comprising
a lipophilic drug moiety having a molecular weight of at least 400; and
a transport moiety, said transport moiety comprising
a hydrophobic spacer with a length of at least 3 atoms and less than 18 atoms chemically linked to said lipophilic drug moiety, and
at least one hydrophilic group chemically linked to said hydrophobic spacer, and
spaced away from said lipophilic drug moiety by said hydrophobic spacer;
wherein said at least one hydrophilic group comprises at least one ionizable atom with a pKa of 4.5 or less;
and wherein said lipophilic drug moiety is not a dipeptide compound which is an α-aminocarboxamide containing a 3-amino-2-hydroxy-4-substituted-phenylbutanoyl with a five membered ring connected via an amide bond.

2. The prodrug of claim 1, wherein said hydrophobic spacer is selected from the group consisting of: a substituted or unsubstituted branched or unbranched saturated alkyl chain, a substituted or unsubstituted branched or unbranched unsaturated alkyl chain, a hydrophobic chain comprising at least one substituted or unsubstituted aryl group, and a hydrophobic chain comprising at least one substituted or unsubstituted cycloalkyl group.

3. The prodrug of claim 2, wherein said substituted saturated alkyl chain or said substituted unsaturated alkyl chain comprises an atom or atom group selected from the group consisting of S, O and C═O.

4. The prodrug of claim 1, wherein said lipophilic drug moiety is chemically linked to said hydrophobic spacer of said transport moiety via a bond selected from the group consisting of: an ester, an amide, and a carbonate.

5. The prodrug of claim 1, wherein a chemical linkage between said lipophilic drug moiety and said hydrophobic spacer is hydrolyzable.

6. The prodrug of claim 1, wherein said lipophilic drug moiety has a LogP value of 1.5 or greater.

7. The prodrug of claim 1, wherein said at least one hydrophilic group is COOH or COO⁻ at physiological pH.

8. The prodrug of claim 1, wherein said transport moiety is selected from the group consisting of moieties of the following acids: succinic, diglycolic, thiodiglycolic, fumaric, muconic, adipic, thiodipropionic, 2-ketoglutaric, 3-ketoglutaric, cyclohexanedioylic, glycerosuccinic, citrosuccinic, malosuccinic, 3,3′-oxydipropionic, 4-carboxybenzoic, tetramethylheptanedioic, cis-aconitic, furandicarboxylic, thiodiacetic acid sulfoxide, dihydromuconic, pimelic, glutaric, suberic, sebacic, tetrahydrofuran 2,5-dicarboxylic acid, norcamphoric acid, cyclopentadiene-1,3-dicarboxylic acid and variants of the above having one or more methyl or ethyl branches located between the two carboxylic acid groups.

9. The prodrug of claim 1, wherein said hydrophobic spacer is of a length selected from the group consisting of 17, 16, 15, 14, 13, 12, 11, 10, 9, 8, 7, 6, 5, 4 or 3 atoms.

10. The prodrug of claim 1, wherein said lipophilic drug moiety is an HIV protease inhibitor.

11. The prodrug of claim 10, wherein said HIV protease inhibitor is selected from the group consisting of lopinavir, ritonavir, saquinavir, nelfinavir, atazanavir, indinavir, tipranavir, darunavir, amprenavir, brecanavir (GW640385), mozenavir (DMP450), CTP-518, TMC310911, L-756423, PPL-100 (MK8122), RO033-4649, and SP1256.

12. The prodrug of claim 1, wherein said lipophilic drug moiety is a steroid selected from the group consisting of an estrogen, a progestin, an androgen, a corticosteroid, estradiol, 2-methoxyestradiol, ethynylestradiol, testosterone, cortisol, mestranol, hydroxyprogesterone, medroxyprogesterone estradiol, 2-methoxyestradiol, and ethynylestradiol.

13. The prodrug of claim 1, wherein said lipophilic drug moiety is selected from the group consisting of temozolomide, sorafenib, erlotinib, gefitinib, imatinib, pazopanib, rapamycin, raloxifene, lasofoxifene, basedoxifene, resveratrol, curcumin, etoposide, camptothecin, CPT-11, topotecan, irinotecan, exatecan, lurtecan, DB67, BNP1350, ST1481, CKD602, paclitaxel, docetaxel, vincristine, vinblastine, fingolimod, raltegravir, elvitegravir, MK-2408, lersivirine, daunorubicin, doxorubicin, epirubicin, and idarubicin, and modifications of any of these which conceal hydrophilic groups.

14. The prodrug of claim 13 wherein said lipophilic drug moiety includes concealed hydrophilic groups selected from —OH and —NH— by formation of hydrolysable ester or amide bonds.

15. The prodrug of claim 1 wherein said lipophilic drug moiety is selected from the group consisting of taxanes, anthracyclines, and camptothecin analogues.

16. The prodrug of claim 1 wherein said transport moiety changes a three dimensional structure of an unmodified portion of said lipophilic drug moiety.

17. The prodrug of claim 1 wherein said prodrug is 3, etoposide acetonide hemiglutarate.

18. A method of delivering a lipophilic drug to a cell or tissue, comprising the step of providing to said cell or tissue a prodrug, said prodrug comprising
a lipophilic drug moiety having a molecular weight of at least 400; and
a transport moiety, said transport moiety comprising
a hydrophobic spacer with a length of at least 3 atoms and less than 18 atoms chemically linked to said lipophilic drug moiety, and
at least one hydrophilic group chemically linked to said hydrophobic spacer, and spaced away from said lipophilic drug moiety by said hydrophobic spacer.

19. The method of claim 18, wherein said hydrophobic spacer is selected from the group consisting of: a substituted or unsubstituted branched or unbranched saturated alkyl chain, a substituted or unsubstituted branched or unbranched unsaturated alkyl chain, a hydrophobic chain comprising at least one substituted or unsubstituted aryl group, and a hydrophobic chain comprising at least one substituted or unsubstituted cycloalkyl group.

20. The method of claim 19, wherein said substituted saturated alkyl chain or said substituted unsaturated alkyl chain comprises an atom or atom group selected from the group consisting of S, O and C═O.

21. The method of claim 18, wherein said lipophilic drug moiety is chemically linked to said hydrophobic spacer of said transport moiety via a bond selected from the group consisting of: an ester, an amide, and carbonate.

22. The method of claim 18, wherein a chemical linkage between said lipophilic drug moiety and said hydrophobic spacer is hydrolyzable.

23. The method of claim 18, wherein said lipophilic drug moiety has a LogP value of 1.5 or greater.

24. The method of claim 18, wherein said at least one hydrophilic group is COOH or COO⁻at physiological pH.

25. The method of claim 18, wherein said transport moiety is selected from the group consisting of moieties of the following acids: succinic, diglycolic, thiodiglycolic, fumaric, muconic, adipic, thiodipropionic, 2-ketoglutaric, 3-ketoglutaric, cyclohexanedioylic, glycerosuccinic, citrosuccinic, malosuccinic, 3,3′-oxydipropionic, 4-carboxybenzoic, tetramethylheptanedioic, cis-aconitic, furandicarboxylic, thiodiacetic acid sulfoxide, dihydromuconic, pimelic, glutaric, suberic, sebacic, dodecanedoic, tetrahydrofuran 2,5-dicarboxylic acid, norcamphoric acid, cyclopentadiene-1,3-dicarboxylic acid and variants of the above having one or more methyl or ethyl branches located between the two carboxylic acid groups.

26. The method of claim 18, wherein said hydrophobic spacer is of a length selected from the group consisting of 17, 16, 15, 14, 13, 12, 11, 10, 9, 8, 7, 6, 5, 4 or 3 atoms.

27. The method of claim 18, wherein said lipophilic drug moiety is an HIV protease inhibitor.

28. The method of claim 27, wherein said HIV protease inhibitor is selected from the group consisting of lopinavir, ritonavir, saquinavir, nelfinavir, atazanavir, indinavir, tipranavir, darunavir, amprenavir, brecanavir (GW640385), mozenavir (DMP450), CTP-518, TMC310911, L-756423, PPL-100 (MK8122), RO033-4649, and SP1256.

29. The method of claim 18, wherein said lipophilic drug moiety is a steroid selected from the group consisting of an estrogen, a progestin, an androgen, a corticosteroid, estradiol, 2-methoxyestradiol, ethynylestradiol, testosterone, cortisol, mestranol, hydroxyprogesterone, medroxyprogesterone estradiol, 2-methoxyestradiol, and ethynylestradiol.

30. The method of claim 18, wherein said lipophilic drug moiety is selected from the group consisting of temozolomide, sorafenib, erlotinib, gefitinib, imatinib, pazopanib, rapamycin, raloxifene, lasofoxifene, basedoxifene, resveratrol, curcumin, etoposide, camptothecin, CPT-11, topotecan, irinotecan, exatecan, lurtecan, DB67, BNP1350, ST1481, CKD602, paclitaxel, docetaxel, vincristine, vinblastine, fingolimod, raltegravir, elvitegravir, MK-2408, lersivirine, daunorubicin, doxorubicin, epirubicin, and idarubicin, and modifications of any of these which conceal hydrophilic groups.

31. The method of claim 18, wherein said prodrug is taken into said cell or tissue by a fatty acid transport system.

32. A method of treating a subject in need thereof, comprising the step of administering to said subject said prodrug comprising
a lipophilic drug moiety having a molecular weight of at least 400; and
a transport moiety, said transport moiety comprising
a hydrophobic spacer with a length of at least 3 atoms and less than 18 atoms chemically linked to said lipophilic drug moiety, and
at least one hydrophilic group chemically linked to said hydrophobic spacer, and spaced away from said lipophilic drug moiety by said hydrophobic spacer.

33. The method of claim 32, wherein said subject is immunocompromised.

34. The method of claim 33, wherein said subject suffers from an HIV infection.

35. The method of claim 32, wherein said subject is indicated for multidrug resistance.

36. The method of claim 35, wherein said multidrug resistance is to a cancer drug or to a seizure drug.
